(19) United States
(12) Patent Application Publication
(10) Pub. No.: US 2020/0094070 A1
(43) Pub. Date: Mar. 26, 2020

(54) METHOD AND APPARATUS FOR PHOTOACTIVATING NUCLEAR RECEPTORS

(71) Applicant: THERALASE BIOTECH INC., Wilmington, DE (US)

(72) Inventor: Arkady MANDEL, Toronto (CA)

(21) Appl. No.: 16/608,558

(22) PCT Filed: Apr. 25, 2018
(86) PCT No.: PCT/US2018/029336

§ 371 Date: Oct. 25, 2019

Related U.S. Application Data
(60) Provisional application No. 62/489,747, filed on Apr. 25, 2017.

(51) Int. Cl. A61N 5/06; A61K 33/243; A61K 31/495; A61P 35/00; A61K 31/57; A61K 31/573; A61N 5/10; A61N 2/00; A61N 7/00

(52) U.S. Cl. CPC A61N 5/062; A61K 33/243; A61K 31/495; A61P 35/00; A61N 2005/067; A61K 31/573; A61N 5/10; A61N 2/002; A61N 7/00; A61K 31/57

(57) ABSTRACT

A method of activating a nuclear receptor in an organism, includes the step of irradiating the nuclear receptor with light effective to activate the nuclear receptor to cause a biological effect in the organism; wherein, the nuclear receptor is not specific to a retina of the organism. The biological effect can be, i.e.: an influence exerted on: (a) a hypothalamic-pituitary-adrenal pathway, (b) a renin-angiotensin pathway, (c) a vagal-neuronal pathway, (d) a neuro-hormonal-immunal pathway, or (e) cellular metabolism. Other non-limiting examples of the biological effect include an alteration of the Warburg effect in the organism or an alteration of the response of the organism to ionizing radiation therapy, Photo Dynamic Therapy, surgical interventions, chemotherapy or sonodynamic therapy. The invention also encompasses the use of a laser device to perform the method and a laser device configured to perform the method.

### BACKGROUND OF THE INVENTION

### 1. Field of Invention

This invention relates to the modification of biological processes, and more particularly to the use of laser and/or non-coherent (monochromatic and/or polychromatic) light alone or in combination with other stimuli to modify biological processes of living organisms.

### 2. Description of Related Art

Low Level Laser Therapy (“LLLT”) is a treatment modality used to modulate various biological processes by bio-photomodulative (bio-regulative) and/or photostimulatory effects. LLLT has been shown to have a non-thermal, biomodulative effect on biological tissues, producing beneficial clinical effects in the treatment of neurological, musculoskeletal, and joint conditions. LLLT is non-invasive (and/or minimally invasive) and avoids the potential side effects of drug therapy. More specifically, LLLT is able to deliver photons to skin and targeted tissue, penetrating the layers of skin to reach internal tissues to produce specific, non-thermal photochemical and/or photophysical effects at the subcellular and cellular levels.

It has been found that light can stimulate or suppress (i.e.: modulate) a growing list of genes, oncogenes, cellular proteins, cytokines, Cytochrome C Oxidase (“CcO”), Nitric Oxide (“NO”) and Reactive Oxygen Species (“ROS”). In addition, there is increasing evidence that LLLT of certain characteristics may be able to control subtle, yet powerful mechanisms that could help in the treatment of cancer and other complex diseases. CcO is a key photo-receptor of phototherapeutic modalities (i.e.: red and Near-Infra Red (“NIR”) light), causing increased electron transport, higher mitochondrial respiration and Adenosine TriPhosphate (“ATP”) synthesis. Increased ATP levels and mitochondrial membrane potential modulates the activity of more than 110 genes and stimulate the upregulation of genes coding for subunits of enzymes involved in more ATP synthesis leading to a positive feedback loop.

Like LLLT, Photo Dynamic Therapy (“PDT”) comprises the use of light to treat diseases and undesirable conditions in biological organisms. PDT typically includes the use of Photo Dynamic Compounds (“PDCs”) also known as Photo Sensitizers (“PSs”) to enhance the therapeutic effects of light. See, e.g., U.S. Patent Application Publications Nos. 20170043179 A1, 20170042976 A1, 20160229878 A1, 20160206653 A1, 20160039854 A1 and 20130331367 A1. PDT is currently an active area of research for the treatment of diseases associated with unwanted and/or hyperproliferating cells such as cancer and non-malignant lesions. PDT has also found use in other contexts, including but not limited to the treatment of: acne, psoriasis, proliferative non-malignant conditions, ulcers, wounds and infections, and for sterilization of inanimate objects.

Selected patent publications relating to treatments comprising the use of light include: U.S. Patent Application Publication No. 20120310309 A1 (device and method for treating cancer and other conditions responsive to polarized light and/or laser energy), U.S. Pat. No. 8,651,111 (use of electromagnetic radiation such as light to photobiomodulate the activity of living cells to delay, diminish, retard or even reverse the structural and functional effects of aging of the skin and other living cells and tissues), U.S. Patent Application Publication No. 20150112411 A1 (handheld LLLT device said to reduce the deleterious impact of radiotherapy, and increase the quality of life of patients with head and neck cancer), and U.S. Pat. No. 6,413,267 (method for irradiating a tissue surface of the patient with at least one laser beam, and noninvasively determining in real-time the irradiance and/or radiant exposure of a target tissue at a predetermined depth below the tissue surface by detecting the radial dependence of light remitted from the tissue surface).

Despite the foregoing developments, it is desired to further elucidate the mechanisms by which light and other treatment modalities modulate biological processes and to develop more precisely targeted means for treating diseases and undesirable conditions in living organisms.

All references cited herein are incorporated herein by reference in their entireties.

### BRIEF SUMMARY OF THE INVENTION

A first aspect of the invention is a method of activating a nuclear receptor in an organism, said method comprising irradiating the nuclear receptor with light effective to activate the nuclear receptor to cause a biological effect in the organism; wherein the nuclear receptor is not specific to a retina of the organism.

In certain embodiments, the biological effect is an influence exerted on at least one pathway selected from the group consisting of a hypothalamic-pituitary-adrenal pathway, a renin-angiotensin pathway, a vagal-neuronal pathway and a neuro-hormonal-immunal pathway.

In certain embodiments, the biological effect is an influence on cellular metabolism.

In certain embodiments, the biological effect is an alteration in a response of the organism to a different treatment modality.

In certain embodiments, the different treatment modality is selected from the group consisting of ionizing radiation therapy, PDT, surgical intervention, chemotherapy and sonodynamic therapy.

In certain embodiments, the biological effect is an alteration of a Warburg effect (the observation that most cancer cells predominantly produce energy by a high rate of glycolysis followed by lactic acid fermentation in the cytosol rather than by a comparatively low rate of glycolysis followed by oxidation of pyruvate in mitochondria as in most normal cells) so as to make cells more susceptible to treatment and/or prophylaxis of a disorder.

In certain embodiments, the disorder is selected from the group consisting of a mitochondrial disorder, a cardiovascular disorder, a metabolic disorder, an inflammatory disorder, an immune disorder, a degenerative disorder, muscle fatigue, an aging disorder and cancer.

In certain embodiments, the nuclear receptor is not RNR or NR2E3.

In certain embodiments, the biological effect is treating or preventing a disorder in the organism, which is a mammal.

In certain embodiments, the light is laser light applied in at least one mode selected from the group consisting of a continuous wave mode, a pulsed mode or a superpulsed mode.

In certain embodiments, the light has a photon energy less than 10 eV.

In certain embodiments, the light has a photon energy of 9.99-1.23 eV.

In certain embodiments, the light has a photon energy of 1.23-0.411 eV.

In certain embodiments, 10-90% of photons of the light have a photon energy of 9.99-1.23 eV.

In certain embodiments, 10-90% of photons of the light have a photon energy of 1.23-0.411 eV.

In certain embodiments, the light is administered at frequencies at or above 10,000 Hz or below 10,000 Hz.

In certain embodiments, the light is applied in a patient-specific protocol.

Certain embodiments further comprise administering to the organism at least one member selected from the group consisting of X-rays, magnetic stimuli, electrical stimuli, ultrasound and medicaments. In such embodiments, the medicaments are preferably PDCs, cytotoxic drugs, steroids, hormones, sirolimus, tacrolimus, anti-inflammatories, immunomodulators, vitamins or vitamin analogues.

In certain embodiments, light of specific wavelengths, modulations and/or energy doses influences the functioning of nuclear receptors in a biphasic response, which in turn positively or negatively affects reactions of the organism to stimuli.

In certain embodiments, the biological effect mimics an effect of a naturally occurring hormone or a synthetic hormone.

In certain embodiments, the biological effect mimics an effect of at least one of sirolimus and tacrolimus.

In certain embodiments, the light induces or represses gene expression.

In certain embodiments, the biological effect is a non-retinal regulation of circadian rhythm.

In certain embodiments, the biological effect is regulation of at least one of calcium and phosphate levels.

In certain embodiments, the biological effect mimics an effect of Peroxisome proliferator-activated receptor (“PPAR”) γ modulators.

In certain embodiments, the biological effect is regulation of at least one of bone homeostasis, bone formation, tissue remodeling and tissue repair.

In certain embodiments, the light is a combination of visible and NIR light and is applied to induce or avoid a biphasic effect.

A second aspect of the invention comprises the use of a laser device to perform the inventive method.

A third aspect of the invention is a laser device configured to perform the inventive method.

### BRIEF DESCRIPTION OF SEVERAL VIEWS OF THE DRAWINGS

The invention will be described in conjunction with the following drawings, wherein:

FIGS. 1A, 1B, 2A, 2B, 3A and 3B are plots of relative fluorescence against the dose of light administered at 905 nm, the dose of light administered at 660 nm and the dose of light administered by combined administration at 905 nm and 660 nm.

FIGS. 4A, 4B, 4C, 4D, 5A, 5B, 5C, 5D, 5E, 6A, 6B, 6C, 7A, 7B, 7C, 7D, 7E, 8A, 8B, 8C, 8D, 9A and 9B are plots of relative mRNA expression against the dose of light administered at 905 nm, the dose of light administered at 660 nm and the dose of light administered by combined administration at 905 nm and 660 nm.

FIG. 10A is a plot of the relative glutamine/glutamate ratio against the dose of light administered at 905 nm, the dose of light administered at 660 nm and the dose of light administered by combined administration at 905 nm and 660 nm.

FIG. 10B is a plot of the relative glutamate concentration against the dose of light administered at 905 nm, the dose of light administered at 660 nm and the dose of light administered by combined administration at 905 nm and 660 nm.

FIG. 10C is a plot of the relative glutamine/glutamate ratio against the dose of light administered at 905 nm, the dose of light administered at 660 nm and the dose of light administered by combined administration at 905 nm and 660 nm.

FIG. 10D is a plot of the relative glutamate concentration against the dose of light administered at 905 nm, the dose of light administered at 660 nm and the dose of light administered by combined administration at 905 nm and 660 nm.

FIG. 10E is a plot of relative glutamine concentration against the dose of light administered at 905 nm, the dose of light administered at 660 nm and the dose of light administered by combined administration at 905 nm and 660 nm.

FIGS. 11A and 11B are plots of Extra Cellular Acidification Rate (“ECAR”) against the dose of light administered at 905 nm, the dose of light administered at 660 nm and the dose of light administered by combined administration at 905 nm and 660 nm.

FIGS. 11C and 11D are plots of Oxygen Consumption Rate (“OCR”) the dose of light administered at 905 nm, the dose of light administered at 660 nm and the dose of light administered by combined administration at 905 nm and 660 nm.

FIGS. 12A, 12B, 12C, 12D, 13A, 13B, 14A, 14B, 14C, 14D and 15 are bar graphs of cell kill as a function of LLLT conditions.

FIGS. 16, 17 and 18 are plots of percent survival against time.

FIG. 19A is a plot of absorbance against wavelength.

FIG. 19B is a plot of percent of photons absorbed against wavelength.

FIGS. 20A and 20B are bar graphs of fold change as a function of LLLT conditions.

FIGS. 21A and 21B are plots of relative mRNA expression against the dose of light administered at 905 nm, the dose of light administered at 660 nm and the dose of light administered by combined administration at 905 nm and 660 nm.

FIGS. 22A, 22B, 22C and 22D are bar graphs of cell kill as a function of LLLT conditions in the presence and absence of Mifepristone.

FIGS. 23A, 23B, 23C and 23D are bar graphs of cell kill by Cisplatin as a function of LLLT conditions in the presence and absence of Mifepristone.

FIGS. 24A, 24B, 24C and 24D are bar graphs of cell kill as a function of LLLT conditions in the presence and absence of Dexamethasone.

FIGS. 25A, 25B, 25C and 25D are bar graphs of cell kill by Cisplatin as a function of LLLT conditions in the presence and absence of Dexamethasone.

FIGS. 26A, 26B, 26C, 26D, 26E, 26F, 26G, 26H, 26I and 26J are bar graphs of relative mRNA expression in response to LLLT conditions in the presence and absence of Mifepristone or Dexamethasone.

FIGS. 27A, 27B, 27C, 27D, 27E, 27F, 27G and 27H are bar graphs of relative mRNA expression in response to LLLT conditions in response to the presence and absence of Mifepristone or Dexamethasone.

FIG. 28 is a graph of cell kill against the log of concentration of TLD1433.

### DETAILED DESCRIPTION OF PREFERRED EMBODIMENTS OF THE INVENTION

### Glossary

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from the group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

As used herein, the expression “Photo Dynamic Therapy or PDT” refers to a treatment for destroying cells and tissue through use of a drug that can be activated by light of a certain wavelength and dose.

As used herein, the term “Photo Dynamic Compound or PDC” shall mean a compound that provides PDT.

In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

Method of Activating Nuclear Receptors

The invention is based in part on the surprising discovery that nuclear receptors which are not specific to the retina of an organism can be activated by light.

A nuclear receptor specific to the retina is defined herein as a nuclear receptor that is normally found only in retinal and/or ocular tissue. Nuclear receptors specific to the retina include NR1B1, NR1B2, NR1B3 and NR2E3.

Nuclear receptors not specific to the retina that are within the scope of the invention include but are not limited to: NR1A1 (Thyroid hormone receptor-α), NR1A2 (Thyroid hormone receptor-β), NR1C1 (Peroxisome proliferator-activated receptor-α), NR1C2 (Peroxisome proliferator-activated receptor-β/δ), NR1C3 (Peroxisome proliferator-activated receptor-γ), NR1D1 (Rev-ErbAα), NR1D2 (Rev-ErbAα), NR1F1 (RAR-related orphan receptor-α), NR1F2 (RAR-related orphan receptor-β), NR1F3 (RAR-related orphan receptor-γ), NR1H3 (Liver X receptor-α), NR1H2 (Liver X receptor-β), NR1H4 (Farnesoid X receptor), NR1H5 (Farnesoid X receptor-β), NR1I1 (Vitamin D receptor), NR1I2 (Pregnane X receptor), NR1I3 (Constitutive androstane receptor), NR1X1, NR1X2, NR1X3, NR2A1 (Hepatocyte nuclear factor-4-α), NR2A2 (Hepatocyte nuclear factor-4-γ), NR2B1 (Retinoid X receptor-α), NR2B2 (Retinoid X receptor-β), NR2B3 (Retinoid X receptor-γ), NR2C1 (Testicular receptor 2), NR2C2 (Testicular receptor 4), NR2E1 (Homologue of the Drosophila tailless gene), NR2F1 (Chicken ovalbumin upstream promoter-transcription factor I), NR2F2 (Chicken ovalbumin upstream promoter-transcription factor II), NR2F6 (V-erbA-related), NR3A1 (Estrogen receptor-α), NR3A2 (Estrogen receptor-β), NR3B1 (Estrogen-related receptor-α), NR3B2 (Estrogen-related receptor-β), NR3B3 (Estrogen-related receptor-γ), NR3C1 (Glucocorticoid receptor or “GCR”), NR3C2 (Mineralocorticoid receptor), NR3C3 (Progesterone receptor), NR3C4 (Androgen receptor), NR4A1 (Nerve Growth factor IB), NR4A2 (Nuclear receptor related 1), NR4A3 (Neuron-derived orphan receptor 1), NR5A1 (Steroidogenic factor 1), NR5A2 (Liver receptor homolog-1), NR6A1 (Germ cell nuclear factor), NR0B1 (Dosage-sensitive sex reversal, adrenal hypoplasia critical region, on chromosome X, gene 1) and NR0B2 (Small heterodimer partner).

The inventive method comprises irradiating a nuclear receptor with light effective to activate (i.e., “photoactivate”) the nuclear receptor. Activation of a nuclear receptor as defined herein means induction of a structural and/or functional change in the nuclear receptor.

Activation of the nuclear receptor directly or indirectly (e.g., via a pathway) results in a direct or indirect biological effect in the organism. A biological effect as defined herein is any measurable change in or on a cell. Such changes include, but are not limited to: changes in cell appearance or structure, changes in cell activity, changes in cellular metabolism, changes in gene expression levels, changes in up regulation or down regulation of proteins or genes, changes in cell protein expression levels, changes in cell protein activity or changes in cell protein stability. The biological effect can also be an alteration in a response of the organism to a treatment modality other than light treatment.

In certain embodiments, the biological effect is an alteration of the Warburg effect so as to make cells more susceptible to treatment and/or prophylaxis of a disorder. For example, the inventive method can be used to modulate Warburg-mediated glycolysis and acidosis.

The biological effect can be exerted on at least one pathway selected from the group consisting of a hypothalamic-pituitary-adrenal pathway, a renin-angiotensin pathway, a vagal-neuronal pathway, a lipid homeostasis (PPARs activation) pathway and a neuro-hormonal-immunological pathway.

The biological effect can be activated in any organism having nuclear receptors. The organism is preferably a mammal and more preferably a human.

The light effective to activate the nuclear receptor may be provided by a variety of light sources. Suitable light sources include but are not limited to: lasers, Light Emitting Diodes (“LEDs”), fiber optics, naturally occurring light sources, bulbs, filaments, chemicals or lamps. The light source can be external to the patient's body with the light applied transdermally, briefly, intermittently or continuously for shorter or longer periods of times. Alternatively, the light source can be placed within the patient's body briefly or intermittently (i.e.: through an endoscope or fiber optic catheter) or for an extended duration (i.e.: as an implant).

Nuclear receptor activating dose parameters can be determined by a person of ordinary skill in the art with an understanding of the dosimetric and biological factors that govern therapeutic variability. See, e.g., Rizvi et al. “PDT Dose Parameters Impact Tumoricidal Durability and Cell Death Pathways in a 3D Ovarian Cancer Model.” Photochemistry and photobiology. 2013; 89(4):942-952.

Factors to be considered include but are not limited to: the quantity of nuclear receptors at the target site, tissue oxygenation, target (e.g., tumor) localization, size, shape, vascular structure, etc. The following table lists PDT parameters to be adjusted and provides preferred, non-exhaustive, values for said parameters (where 1 Joule=1 Watt/1 second).

| PDT Parameter | Value |
|---|---|
| Wavelength (nm) | 123-1000 or 400-970 or 500-970 |
| Fluence (J/cm²) | 0.01 to 100,000 or 1 to 10,000 or 1 to 1,000 |
| Irradiance (mW/cm²) | 10 to 10,000 or 10 to 5,000 or 10 to 1,000 |
| Irradiation Time (secs) | 0.001 to 10,000,000 or 0.2 to 1,000,000 or |
| | 1 to 100,000 |

The light is preferably laser light or LED light applied in at least one mode selected from the group consisting of: continuous wave mode, pulsed mode and superpulsed mode.

In certain embodiments of the invention, a potential biphasic response induced by LLLT is mitigated by activation of nuclear receptor(s) with a combination of visible and/or NIR light, for example 660 nm in pulse mode and 905 nm in superpulse mode.

Photon energy in Joules can be calculated as: E=hf=hc/λ, where: 1 Joule is equal to 6.24150647996×10¹⁸ electron volts, E is photon energy, h is the Planck constant (6.626×10⁻³⁴ J-s), c is the speed of light in a vacuum (3×10⁸ m/s) and λ is the photon's wavelength (as measured in nanometers) or photon energy in electron volts (“eV”) can be calculated as: 12345 divided by the wavelength as measured in angstroms (10⁻¹⁰ meters).

Fluence rates for each wavelength range from 1 to 1000 J/cm². In certain embodiments, the fluence for each wavelength is above 1000 J/cm², with a broad fluence rate, for example 10-1,000 mW/cm², but below the thermal effect at temperatures above 41° C. (105.8° F.).

The discovery of the role played by nuclear receptor activation in the biological effects of exposing cells and organisms to light makes it possible to more precisely administer the light so as to achieve a desired effect and minimize undesirable side effects.

In certain embodiments, treatment (e.g., light wavelength(s), energy doses, pulse sequence, modes, etc.) can be personalized for an individual.

Combination Therapies

Light effective to activate nuclear receptors can be administered alone and/or before and/or after the administration of other treatment modalities. Treatment modalities suitable for combining with light therapy according to the invention include but are not limited to: PDT, ionizing radiation therapy, chemotherapy, surgical interventions, anti-cancer therapy, immunomodulatory therapy, other systemic or local therapeutic modalities, antineoplastic drugs and/or sonodynamic therapy.

In certain embodiments, the additional treatment modalities may act as agonists to activate the nuclear receptor(s), to achieve additive effects to LLLT, and/or may work independently via different nuclear receptor(s) mechanisms/pathways to synergistically modulate: cellular metabolism, pH and/or acidosis and/or gene expression.

In certain embodiments, the photoactivation of nuclear receptors preconditions tumor cells and tissues to become more susceptible to anti-cancer therapies, including but not limited to: PDT, ionizing radiation therapy, chemotherapy, surgical interventions, immunomodulatory therapy, other systemic or local therapeutic modalities antineoplastic drugs and/or sonodynamic therapy.

Preferred additional treatment modalities are disclosed in WO 2013158550 A1, WO 2014145428 A2, U.S. Pat. Nos. 6,962,910, 7,612,057, 8,445,475, 8,148,360, US 20160206653 A1 and U.S. Application 62/325,226, filed Apr. 20, 2016.

The invention will be illustrated in more detail with reference to the following Examples, but it should be understood that the present invention is not deemed to be limited thereto.

### EXAMPLES

### Example 1: LLLT and ROS Production in the Mitochondria that May Relate to the Treatment of Mitochondria and/or Mitochondrial Disease Related Pathology

Damaged or cancer cells do not have functional mitochondria or their mitochondria may be in a “dormant” quiescent phase. LLLT reactivates the mitochondria oxidative phosphorylation process which leads to ROS production, which is used as a surrogate biomarker of mitochondrial activity.

Rat glioma (“RG2”) cells (30,000 cells) or human bladder cancer (“T24”) cells (15,000 cells) were seeded in complete Dulbecco's Modified Eagle's Medium (“DMEM”) in 96-well culture plates and incubated at 37° C., 5% CO₂ overnight. The next day, the cells were washed once with Phosphate Buffered Saline (“PBS”) and the medium was replaced with complete DMEM without phenol red or sodium pyruvate. Cells were treated with either 660 nm, 905 nm or an equal combination of both wavelengths at fluence doses from 0.5 to 20 J/cm². Immediately after, cells were incubated with a mitochondrial superoxide detector (Mitosox Red, Thermofisher) per the manufacturer's protocol. Cells were then washed with PBS and fluorescence was measured using a plate reader at excitation/emission of 510/580 nm. Values are normalized to control cells that were kept in the dark and not treated with LLLT.

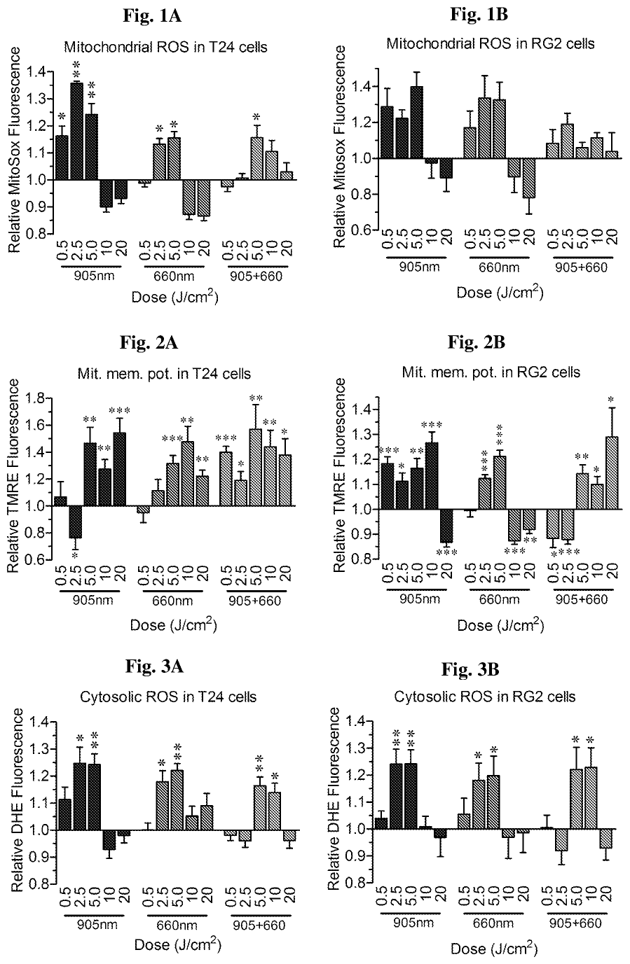

The effect of LLLT on mitochondrial ROS production is shown in FIG. 1A (T24 cells) and FIG. 1B (RG2 cells).

The data revealed that LLLT has a unique “fingerprint” (i.e., energy dose, light combination and/or treatment mode) dependent effect, and can regulate key mitochondrial processes. Mitochondrial ROS production was increased: 1) in the cells treated with 905 nm (pulsed-wave (“PW”)) at the fluence values of 0.5 to 5 J/cm². The higher fluence values at 10 J/cm² and 20 J/cm² inhibited ROS production; hence, may inhibit mitochondrial function; 2) in the cells treated with 660 nm (continuous wave (“CW”)), which exhibited similar dose-dependent effects on mitochondrial ROS production; 3) the combination of 905 nm CW and 660 nm CW was the most effective in the production of ROS by the mitochondria and the LLLT biphasic dose response was not distinct in this treatment group. The data presented in this figure suggest that LLLT has a direct and a particular fingerprint effect on the fundamental molecular mechanisms and can represent an important modulator of cellular function.

### Example 2: LLLT and Mitochondrial Membrane Potential

Mitochondria are the main powerhouse in cells and play important roles in processes such as: steroid metabolism, calcium homeostasis, apoptosis and cellular proliferation. Mitochondrial transmembrane potential is an important parameter of mitochondrial function and is a key indicator of its function and cell health. In many cancer cells, mitochondria may seem dysfunctional, manifested by a shift of energy metabolism from oxidative phosphorylation to active glycolysis, and decreased mitochondrial membrane potential. Importantly, the metabolic reprogramming of mitochondria in a cancer cell is mechanistically linked to oncogenic signals; therefore, a LLLT-mediated increase in mitochondria transmembrane potential could potentially reverse the dysfunction in the mitochondria of cancer cells and inhibit oncogenic signals, which could be used as a cancer therapy strategy.

RG2 cells (30,000 cells) or human bladder cancer (T24) cells (20,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate. Cells were treated with either 660 nm, 905 nm or an equal combination of both wavelengths at fluence doses from 0.5 to 20 J/cm². Immediately after, the cells were incubated with 0.5 uM tetramethylrhodamine, ethyl ester, (“TMRE”; Thermofisher), which is a cell membrane permeable cationic dye that accumulates in the mitochondria in response to their high membrane potential and negative charge. Cells were then washed with PBS and fluorescence was measured using a plate reader at excitation/emission of 550/575 nm. Values were normalized to control cells that were kept in the dark and not treated with LLLT.

The effect of LLLT on mitochondrial membrane potential in RG2 and T24 cells is shown in FIG. 2A and FIG. 2B respectively. The data reveal a similar fingerprint to ROS production in FIGS. 1A and 1B, in which a biphasic response was observed when individual lasers were used; whereas, higher fluence doses seem to be tolerated better and no biphasic response is observed for combined laser treatment. Maximal mitochondrial membrane potential was noted: 1) in the cells treated with 905 nm PW at the fluence value of 0.5 J/cm² to 5 J/cm². The higher fluence value at 20 J/cm² promoted a biphasic suppressive effect; 2) in the cells treated with 660 nm CW in fluence values from 2.5 J/cm² to 5 J/cm², while the higher fluence at 20 J/cm² promoted a biphasic suppressive effect; and 3) the combination of 905 nm PW and 660 nm CW has an up-regulative effect on the production of cytosolic ROS across a majority of the tested doses of LLLT.

### Example 3: LLLT and ROS Production in the Cytosol

The second main source of ROS after mitochondria is cytosolic components such as NADPH oxidases, which catalyze the transfer of electrons from NADPH to molecular oxygen via their catalytic subunit, generating O₂.⁻ and H₂O₂; therefore, it is of importance to reveal the effect of LLLT on cytosolic ROS production.

RG2 cells (30,000 cells) or human bladder cancer (T24) cells (15,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate. Cells were treated with either 660 nm, 905 nm or a combination of both wavelengths at fluence doses from 0.5 to 20 J/cm². Immediately after, cells were incubated with a cytosolic superoxide detector (Dihydroethidium “DHE”; Thermofisher) per the manufacturer's protocol. Cells were then washed with PBS and fluorescence was measured using a plate reader at excitation emissions of 510/580 nm. Values are normalized to control cells that were kept in the dark and not treated with LLLT.

The effect of LLLT on cytosolic ROS production in T24 and RG2 cells is shown in FIGS. 3A and 3B respectively. This data further confirmed that LLLT has a unique “fingerprint” (i.e., energy dose, light combination and/or treatment mode) dependent effect; where, a biphasic response is observed when individual lasers are used independently and the combination of red 660 nm and NIR 905 nm lasers extending the therapeutic range and eliminating the biphasic response. Maximal activation of ROS in the cytosol was noted: 1) in the cells treated with 905 nm PW at the fluence value of 0.5 J/cm² to 5 J/cm². The higher fluence value at 10 J/cm² and 20 J/cm² promoted a biphasic suppressive effect; 2) in the cells treated with 660 nm CW the fluence values from 0.5 J/cm² to 5 J/cm², which reveals the simulative effect on cytosolic ROS production; and 3) the combination of 905 nm PW and 660 nm CW has an up-regulative effect on the production of cytosolic ROS across the tested doses of LLLT.

### Example 4: LLLT Modulates Expression of Genes Related to Cancer, Metabolism and Inflammation

An increase in the ROS levels above a certain threshold (“oxidative stress level”) accompanies processes that are detrimental to cell survival; however, LLLT induced ROS production at low concentrations act as secondary messengers responsible for signal transduction to intracellular regulatory systems and transcription factors, which control gene expression; therefore, this section will focus on the effect of LLLT, as the expression of key transcription factors and genes that play a critical role in cancer development (i.e., oncogenes and/or tumor suppressors), cancer metabolism, Warburg glycolysis and/or cancer inflammation through cytokine production.

RG2 cells (0.5×10⁶ cells) were seeded in complete DMEM in 35 mm culture dishes and incubated at 37° C., 5% CO₂ overnight. The next day, the cells were washed once with PBS and the medium was replaced with complete DMEM without phenol red or sodium pyruvate. Cells were then treated with either 660 nm, 905 nm or a combination of both wavelengths at fluence doses from 0.5 to 20 J/cm². Cells were incubated for 6 hours, then the cells were washed once with PBS and harvested by TRIzol reagent for RNA extraction (Invitrogen) per the manufacturer's protocol. 2 ug of RNA were used to prepare cDNA using the high-capacity cDNA reverse transcription kit (Thermofisher) and the cDNA was diluted 1:10. 2 μl of cDNA was used per PCR reaction using the SYBR green master mix and qPCR was run on the LightCycler 480 (Roche). Gene expression was normalized to the housekeeping gene HPRT, and values were normalized to control cells that were kept in the dark and not treated with LLLT.

The reliance of cells on glycolysis due to chronic inflammation or cancer is associated with the activation of oncogenic pathways. One of the most commonly altered signaling pathways in human cancer is the phosphoinositide 3-kinase (“PI3K”) pathway. Once activated, the PI3K pathway strongly promotes cancer cell proliferation and survival but also affects cell metabolism. The main effector of the PI3K pathway is Akt. Akt is a regulator of glycolysis and plays a major role in the regulation of the bioenergetic balance. It stimulates glycolysis by increasing the expression and translocation of glucose transporters. Finally, Akt is a strong activator of the mechanistic target of Rapamycin (“mTOR”), a key metabolic and bioenergetic checkpoint that integrates growth signaling and nutrient availability. Activated Akt strongly stimulates mTORC1, which positively regulates protein, lipid and nucleotide synthesis in response to sufficient nutrient and energy conditions. mTORC1 activation is a strong anti-apoptotic and pro-survival signal, as well as a key player in the mechanisms of tissue repair and aging. LLLT at specific wavelength and light doses downregulate mTOR expression in malignant cells, which leads to a decrease in glycolysis and reversal of the Warburg effect in cancer cells.

The tumor suppressor p53 is a transcription factor that can either activate or repress the expression of many genes. Additional activities of p53 are mediated by its direct interactions with metabolic enzymes and apoptotic/cell death effectors. P53 maintains metabolic homeostasis and allows cells to adapt to and survive metabolic stress, which can help to prevent damage and limit cancer development. A key metabolic function of p53 is to regulate energy metabolism by lowering the rate of glycolysis and augmenting mitochondrial respiration. p53 downregulates cellular glucose uptake and the expression of other glycolytic enzymes. Many glycolytic enzymes are upregulated in tumors because of elevated c-Myc transcriptional activity and insufficient p53-mediated control; therefore, upregulation of p53 expression can lead to limited Warburg glycolysis in tumor cells and inhibited tumor cell proliferation.

The c-Myc oncogene is a “master regulator” which controls many aspects of both cellular growth and cellular metabolism. The metabolic changes which occur in cancer cells are driven by overexpression of c-Myc and are necessary to support the increased need for nucleic acids, proteins and lipids necessary for rapid cellular proliferation. At the same time, c-Myc overexpression results in coordinated changes in level of expression of gene families which results in increased cellular proliferation. The effects induced by c-Myc can occur either as a “primary oncogene” which is activated by amplification or translocation; or as a downstream effect of other activated oncogenes. There is an urgent need to develop effective inhibitory treatments to target the key inducers of cancer metabolic reprogramming such as c-Myc. Effective and direct inhibition of c-Myc still requires additional study because this protein cannot currently be targeted with drugs. In fact, suppressing glycolysis and glutaminolysis remarkably antagonizes the growth of tumors bearing genetic alterations in c-Myc. LLLT at specific wavelength and light doses downregulates c-Myc expression in malignant cells.

Hypoxia-inducible factor 1-alpha (“HIF1-α”) is considered the master regulator of cellular and developmental response to hypoxia. The dysregulation and overexpression of HIF1A by either hypoxia or genetic alterations have been heavily implicated in cancer biology, as well as several other patho-physiologies; specifically, in areas of vascularization and angiogenesis, energy metabolism, cell survival and/or tumor invasion. The transcription factor HIF-1 plays an important role in cellular response to systemic oxygen levels in mammals and is known to induce transcription of more than 60 genes; including, genes involved in cell proliferation and survival, as well as glucose and iron metabolism. During hypoxia, p53 overexpression is associated with a HIF1-α-dependent pathway to initiate apoptosis; however, loss of function in p53 and overexpression of HIF1A in malignant cells results in an anti-apoptotic phenotype that promotes tumor cell survival; therefore, new therapeutic strategies are required to selectively and effectively target the HIF1-α pathway to decrease tumor progression. LLLT at specific wavelengths and light doses downregulate HIF1-α expression in malignant cells and could be the cause for Warburg effect reversal and decreasing malignant development.

Malignant transformation of cells leads to enhanced glucose uptake and its conversion to lactate, which in turn serves to generate biosynthetic precursors to facilitate the survival of rapidly proliferating cells. Glucose transporters (“GLUT1”) are responsible for glucose uptake and are upregulated in tumor cells to supply glucose for Warburg glycolysis. Both glucose and glutamine are metabolic substrates for lactate production. Extracellular lactate directs the metabolic reprogramming of tumor cells. Accumulation of lactate and the acidification of the intracellular milieu have deleterious consequences for the cells. This is prevented by monocarboxylate transporters (“MCTs”), which transport monocarboxylates (e.g., lactate, pyruvate, and ketone bodies) across the plasma membrane of various cell types. MCTs are necessary for lactate input into cells, and they direct both the influx and the efflux of lactate across the plasma membrane. MCTs and GLUT gene expression are increased in various types of tumors and inhibition of their expression would decrease lactate production and transport between cells and reduce the malignant transformation of cells in response to the acidic environment produced by lactic acid transport. LLLT at specific wavelengths and energy doses are able to downregulate MCT-1 and GLUT1 expression in malignant cells.

As mentioned before, metabolism may determine the biologically malignant behavior of cancer cells. During aerobic glycolysis, glucose is phosphorylated by hexokinase 2 (“HK2”) to form glucose-6-phosphate and lactic acid is produced from pyruvic acid by pyruvate kinase isoenzyme type M2 (“PKM2”). PKM2 and HK2 are glycolytic enzymes that are essential for Warburg effect glycolysis and their inhibition or depletion in cancer cells may restore oxidative glucose metabolism and increase sensitivity to cell death inducers. The peroxisome proliferator-activated receptor gamma (“PPAR-γ2”) transcription factor and nuclear hormone receptor contributes to selective PKM2 and HK2 gene expression regulation. LLLT at specific wavelengths and energy doses is able to downregulate PPAR-γ2 expression, which leads to decreased PKM2 and HK2 expression in tumor cells.

Generally, the effect of LLLT on gene expression is variable and formulating conclusions as to which energy dose, wavelength combination and/or treatment mode is most beneficial is not a direct correlation. In addition, one would need to take into consideration how these effects translate into protein expression by quantifying protein levels through, for example: Western Blots or immunofluorescence microscopy. In some cases, a biphasic response is observed when individual lasers are used independently; whereas, the combination of red 660 nm and NIR 905 nm lasers extends the therapeutic range and may eliminate the biphasic response. In other instances, a uniform effect of light is observed on gene expression regardless of the energy dose, wavelength selection or combination or treatment mode. Finally, in other genes, a biphasic response is observed with different energy doses; however, is independent of whether individual wavelengths or combination wavelength treatment was used.

Regardless, LLLT showed positive effects on gene expression that would yield reversal of Warburg glycolysis, increased mitochondrial respiration, decreased oncogenesis and inflammation, and/or restoration of normal cell metabolism.

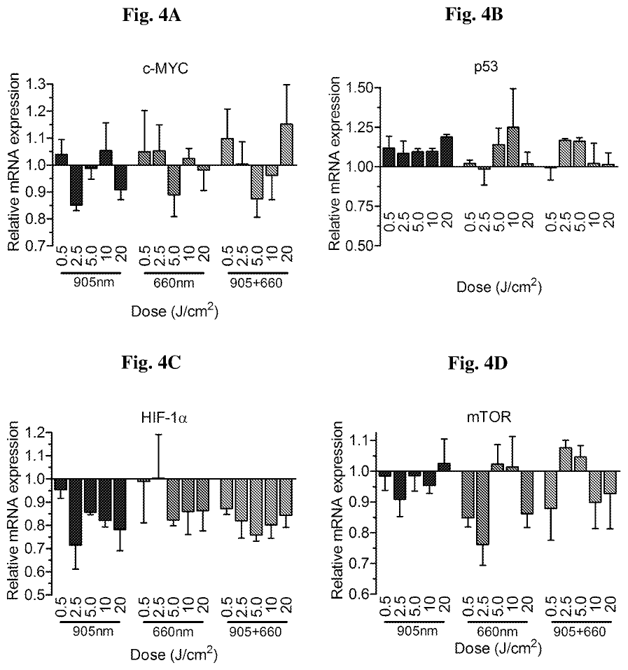

FIGS. 4A-4D show that LLLT modulates the expression of oncogenes and tumor suppression in cancer cells. LLLT at certain doses reduced c-Myc (FIG. 4A), mTOR (FIG. 4D), and HIF-1α expression (FIG. 4C), and increased p53 expression (FIG. 4B), which would suggest less oncogenic activity.

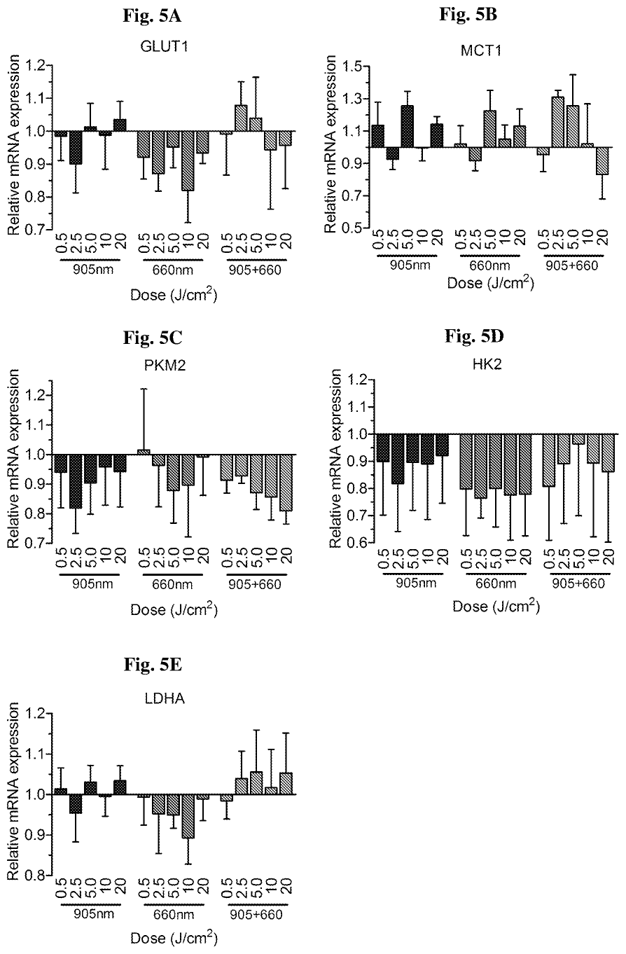

FIGS. 5A-5E show that LLLT modulates the expression of genes related to glycolysis. LLLT reduced the expression of key glycolytic enzymes such as GLUT1 (FIG. 5A), PKM2 (FIG. 5C), HK2 (FIG. 5D) and increased expression of genes that remove lactic acids such as MCT1 (FIG. 5B) and LDHA (FIG. 5E).

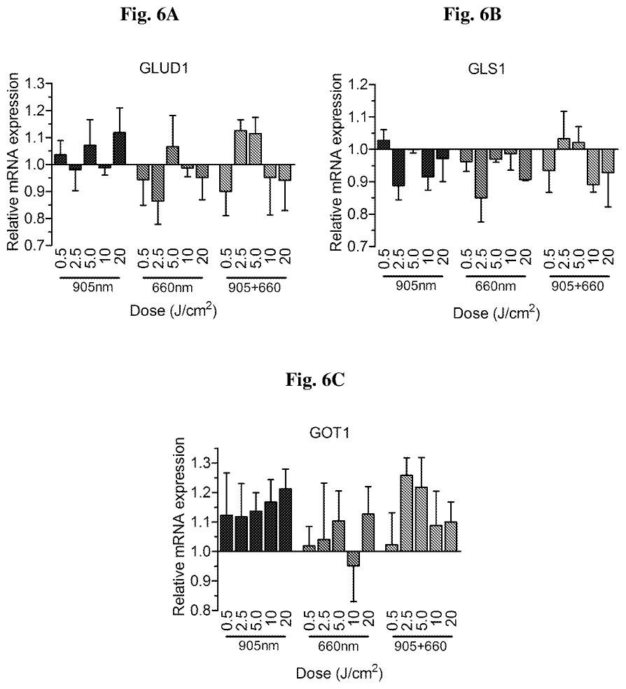

FIGS. 6A-6C show that LLLT modulates the expression of genes related to glutaminolysis, such as GOT1 (FIG. 6C), GLUD (FIG. 6A) and GLS1 (FIG. 6B).

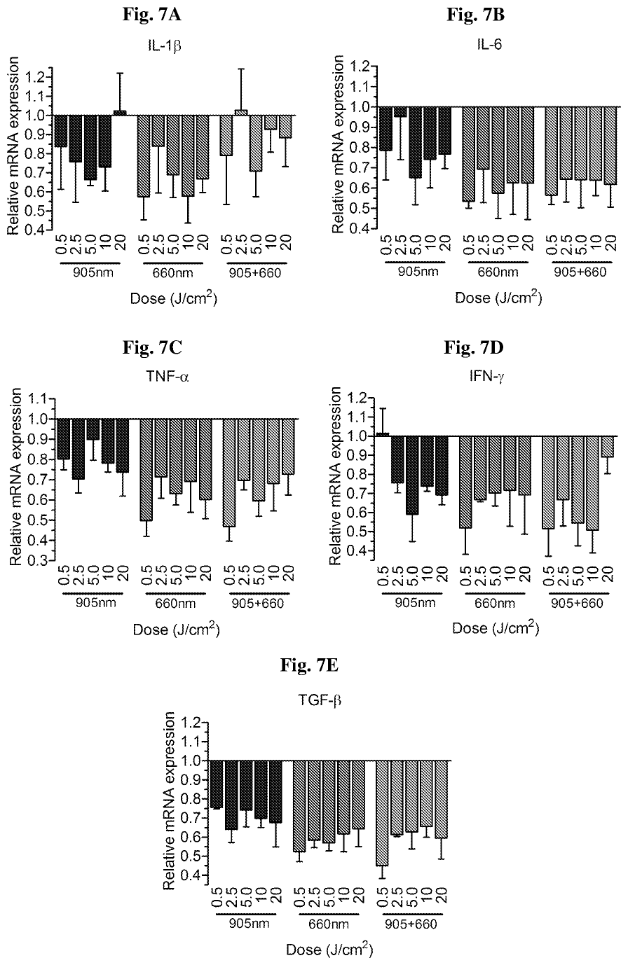

FIGS. 7A-7E show that LLLT modulates inflammatory gene expression in cancer cells, such as IL-1β (FIG. 7A), IL-6 (FIG. 7B), TNF-α (FIG. 7C), IFN-γ (FIG. 7D) and TGF-β (FIG. 7E).

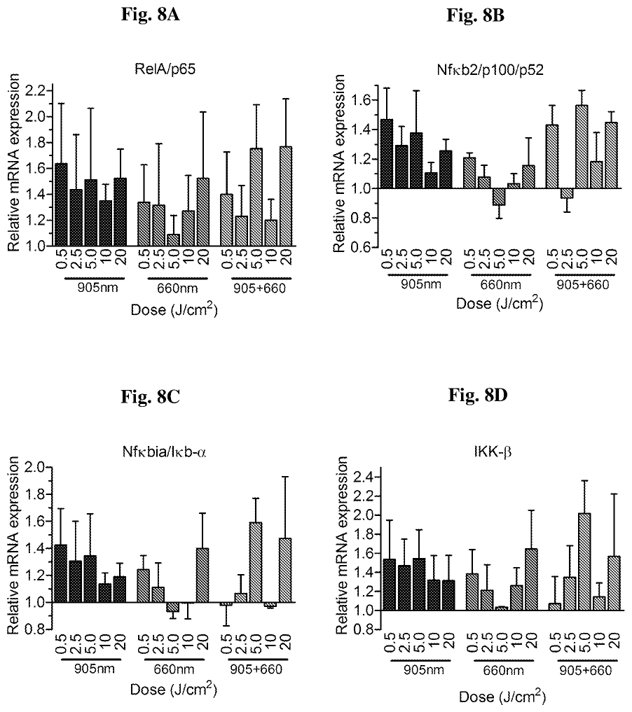

FIGS. 8A-8D show that LLLT modulates expression of NF-κB genes, such as RelA/p65 (FIG. 8A), NF-KB2/p100/p52 (FIG. 8B), NF-KB1A/IκBα (FIG. 8C) and IKK-3 (FIG. 8D), and inhibited inflammatory gene expression. Altogether, this yields beneficial outcomes to repair damaged, stressed, inflamed and/or cancer cells.

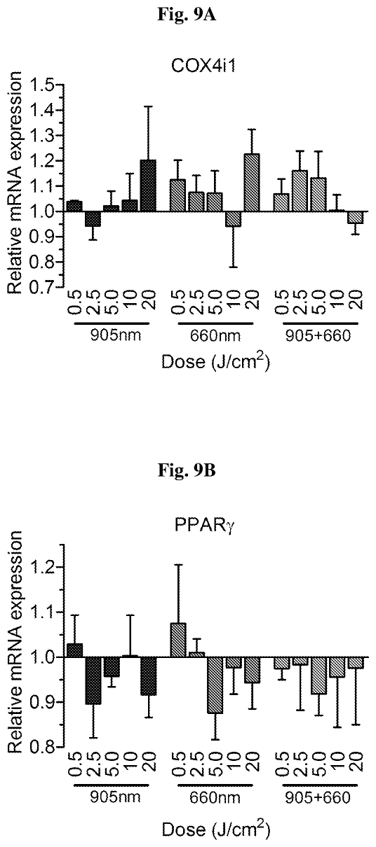

FIGS. 9A-9B show that LLLT modulates expression of other genes, such as COX4i1 (FIG. 9A) and PPARγ (FIG. 9B).

### Example 5: LLLT Modulates Glutaminolysis in Rat Glioma Cells

Brain tumor (glioma) cells rely more on glutamine rather than glucose as a source of energy, via the Warburg effect. Glutaminolysis is a series of biochemical reactions by which the amino acid glutamine is lysed to glutamate. Glutaminolysis takes place in all proliferating cells, especially in tumor cells through the overexpression of the Glutaminase enzymes (“GLS1”) and the inhibition of the glutamate dehydrogenase (“GLUD1”) and is a main pillar for energy production besides glycolysis. In addition, there is a functional link between c-Myc and glutamine metabolism, in which Myc induces a transcriptional program that promotes glutaminolysis in cancer cells. Moreover, the mTOR pathway upregulates GLS1 expression through enhancing the translation of c-Myc protein; therefore, interventions in this metabolic process could provide novel approaches to improve cancer treatment.

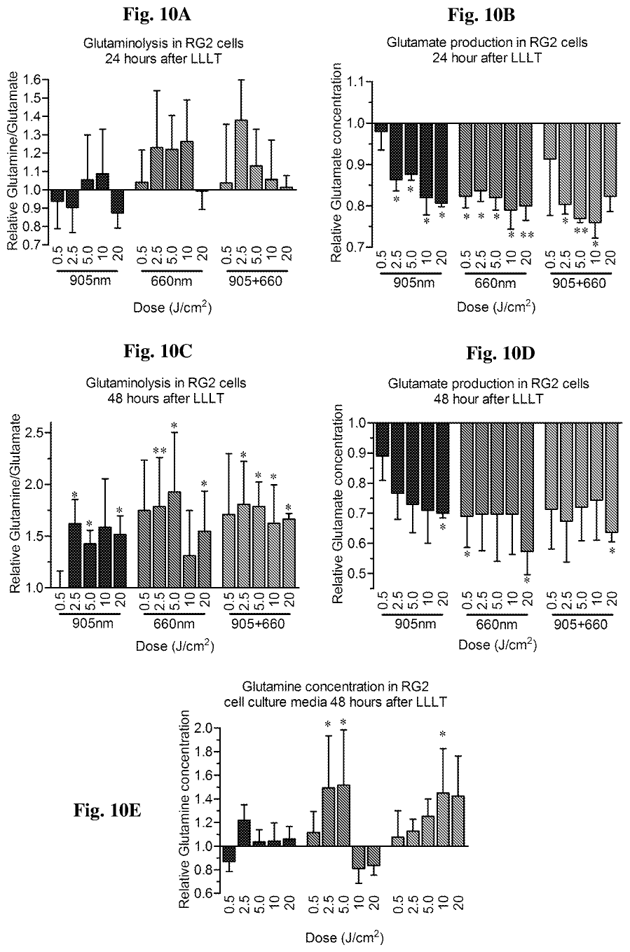

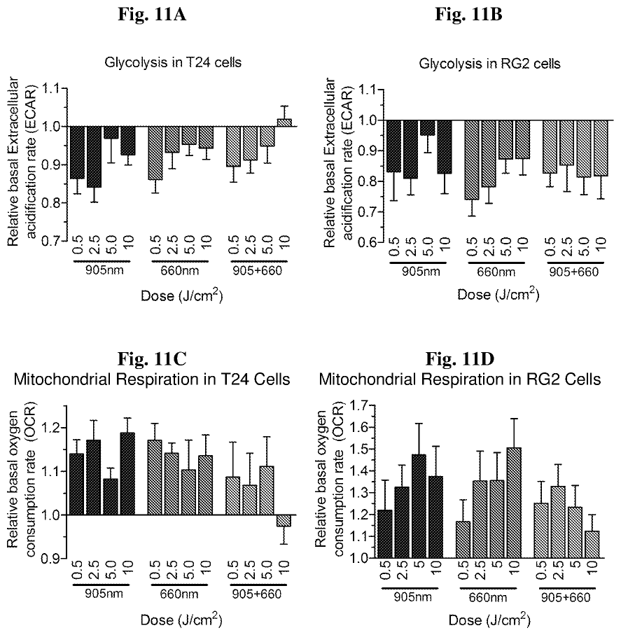

RG2 cells (0.3×10⁶ cells) were seeded in complete DMEM in 35 mm culture dishes and incubated at 37° C., 5% CO₂ overnight. The next day, the cells were washed once with PBS and the medium was replaced with complete DMEM without phenol red or sodium pyruvate. Cells were treated with either 660 nm, 905 nm or a combination of both wavelengths at fluence doses from 0.5 to 20 J/cm². Cells were incubated for 1 or 2 days, then the culture supernatant was collected for metabolic analysis. Glutamine and glutamate concentrations were determined from the cell culture supernatant using the BioProfile 100 Plus Biochemical Analyzer (Nova Biochemical) using 0.5 ml of media. Values were normalized to control cells that were kept in the dark and not treated with LLLT (FIGS. 10A-10E).

The glutamine to glutamate ratio in RG2 glioma cells at 24 and 48 hours after LLLT are shown in FIGS. 10A and 10C, respectively. A higher ratio indicates lower glutaminolysis because of higher glutamine concentration in the media (less glutamine was consumed by the cells), lower glutamate production by the cells or both. Maximal suppression of glutaminolysis was observed: 1) in the cells treated with 905 nm PW at the fluence value of 5 J/cm² and 10 J/cm²; whereas, a biphasic response was observed at the lower and/or higher fluence values; 2) in the cells treated with 660 nm CW the fluence values from 0.5 to 10 J/cm² reveal the beneficial effect on cellular metabolism; and 3) the combination of 905 nm (2.5 J/cm², PW) and 660 nm (2.5 J/cm², CW) was the most effective in suppression of the Warburg effect; where the LLLT biphasic dose response was not distinct in this treatment group, 4) in all conditions after 2 days after LLLT. The increase in glutamine to glutamate ratio was primarily due to a decrease in glutamate production (see FIGS. 10B and 10D); where, a biphasic response was observed in glutamine concentration at 2 days after LLLT with the 905 nm PW and the 660 nm CW individually; whereas, the biphasic response was not observed in the combined 905 nm and 660 nm treatments. (See FIG. 10E).

### Example 6: LLLT Modulates Glycolysis and Oxidative Phosphorylation in Cancer Cells

Cancer cells may rewire their metabolism to promote growth, survival, proliferation, and/or long-term maintenance. The common feature of this altered metabolism is the increased glucose uptake and its associated fermentation into lactate. This phenomenon is observed even in the presence of completely functional mitochondria, and is known as the Warburg effect. In tumor cells, the rate of glucose uptake increases dramatically, and lactate is produced even in the presence of oxygen and fully functioning mitochondria. Therefore, the effect of LLLT on glycolysis and mitochondrial respiration is of importance. Glycolysis and mitochondrial respiration were measured using Seahorse XF technology, which measures cellular metabolism in live cells, via the measurement of ECAROCR to provide insights into glycolytic activity and mitochondrial respiration. During glycolysis, glucose is converted to lactic acid, which is transported outside the cells and results in increases in acidification of the extracellular environment, correspondingly, decreasing glycolysis reduces the extracellular acidification rate. During mitochondrial respiration, oxygen is being consumed, and increased oxygen consumption indicates activation of the mitochondria from a “dormant” quiescent state to an “active” energetic state.

RG2 cells (30,000 cells) or human bladder cancer (T24) cells (20,000 cells) were seeded in complete DMEM in 96-well Seahorse culture plates (Agilent technologies) and incubated at 37° C., 5% CO₂ overnight. The next day, the cells were washed once with PBS and the medium was replaced with complete DMEM without phenol red or sodium pyruvate. Cells were treated with either 660 nm, 905 nm or a combination of both wavelengths at fluence doses from 0.5 to 10 J/cm². Cells were incubated for 24 hours. The media was replaced with XF media without sodium bicarbonate and the plate was placed at 37° C. in XF prep station (0% CO₂) for 1 hour. Basal ECAR and OCR levels were measured using a XFe96 analyzer (Agilent technologies) per the manufacturer's protocol. Values were normalized to control cells that were kept in the dark and not treated with LLLT.

The effect of LLLT on ECAR in T24 cells is shown in FIG. 11A. The effect of LLLT on ECAR in RG2 cells is shown in FIG. 11B. The effect of LLLT on OCR in T24 cells is shown in FIG. 11C. The effect of LLLT on OCR in RG2 cells is shown in FIG. 11D.

The data reveal that LLLT changes the metabolic state of cancer cells from a glycolytic state into an aerobic state by decreasing glycolysis (FIGS. 11A and 11B) and increasing mitochondrial respiration rates (FIGS. 11C and 11D), indicating reversal or inhibition of the Warburg effect. This effect was observed at most energy doses, wavelength combinations and/or treatment modes. Furthermore, LLLT did not induce biphasic effects on glycolysis and mitochondrial respiration at the energy doses, wavelength combinations and/or treatment modes tested, which suggests a wider range for therapeutic effects without the need to worry about adverse effects, which would allow for treatment of a larger tumor area and/or volume.

In summary, LLLT induces changes in cancer cells at the level of gene expression and the metabolic state of cells that suggests that LLLT slows down or reverses some of the key oncogenic processes; therefore, the question now arises whether LLLT would render cancer cells more sensitive to other anti-cancer therapies such as PDT, ionizing radiation and/or antineoplastic drugs. This could potentially increase the efficacy of treatment and decrease the toxicity that results from PDT, radiation and/or drugs.

### Example 7: LLLT and PDT

LLLT is primarily used to stimulate healing, reduce/eliminate pain and reduce inflammation and prevent cellular tissues from dying through apoptosis or necrosis. LLLT using varying wavelengths and energy doses has been proven to protect cells in culture from dying after various cytotoxic insults and LLLT is known to increase the cellular ATP content. Previous studies have demonstrated that maintaining a sufficiently high ATP level is necessary for the efficient induction and execution of apoptosis and/or necrosis after PDT.

RG2 cells (15,000 cells) or human bladder cancer (T24) cells (10,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate. Cells were treated with either 660 nm, 905 nm or a combination of both wavelengths at the indicated fluence doses in J/cm². After 6 or 24 hours, the media was replaced with media containing the ruthenium-based PDC 14C (100 nM for RG2 cells and 200 nM for T24 cells) for 1 hour, then the PDC was washed away and the cells were treated with PDT using green laser light (wavelength: 530 nm, fluence 20 J/cm²) using a 96-laser diode array light source (TLD3000, Theralase Inc. Toronto, ON, Canada), after which the plates were returned to the dark in the incubator overnight. Cell viability was measured by Presto Blue cell viability assay quantifying fluorescence at using a plate reader at excitation emissions of 560/600 nm. PDT-mediated cell kill was calculated by subtracting cell kill due to LLLT first and values were normalized to control cells that were kept in the dark and not treated with LLLT.

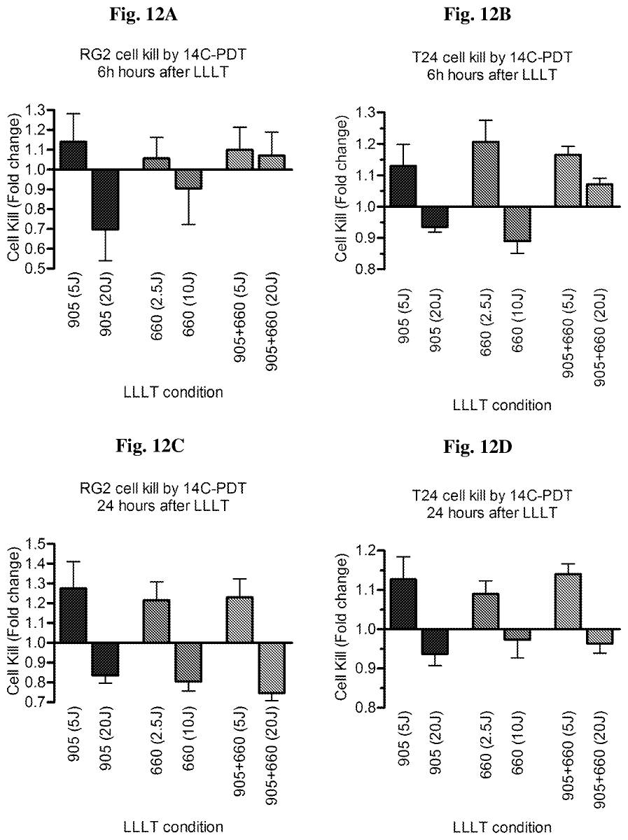

The data shown in FIGS. 12A-12D revealed that LLLT has a biphasic effect on enhancing anti-cancer treatment through PDT. 14C-mediated cell kill was increased in cells treated with 905 nm PW or 660 nm CW at the fluence value of 5 J/cm² and 2.5 J/cm², respectively; whereas, the higher fluence values of 20 and 10 J/cm² promoted cell survival. The combination of 905 nm PW and 660 nm CW was the most effective and a LLLT biphasic response was not distinct at 6 hours post LLLT treatment (FIGS. 12A and 12B). The data represented in FIGS. 12C and 12D suggest that LLLT performed at different timepoints prior to PDT increased the PDT efficacy, most probably by changing the cell signaling pathways and increasing ATP production, which lead to enhanced cellular uptake of the PDC and more efficient apoptosis and/or necrosis induced by the PDC; therefore, LLLT can potentially be used to increase the efficacy of PDT and allow for decreased energy and/or drug doses to minimize toxicity of the conducted anti-cancer therapy.

### Example 8: LLLT and Radiotherapy by Ionizing Radiation (X-ray)

Some tumors are naturally resistant to treatment by ionizing radiation and cannot be treated by radiotherapy. This example shows that reversal of Warburg glycolysis and changing the metabolism and key signaling factors in tumor cells overcomes the resistance to ionizing radiation and improved the sensitivity and cell kill of tumor cells.

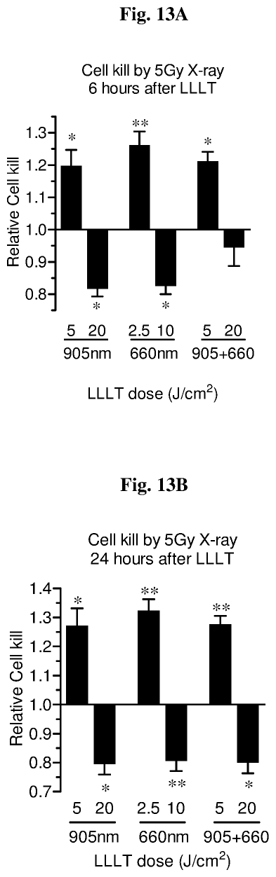

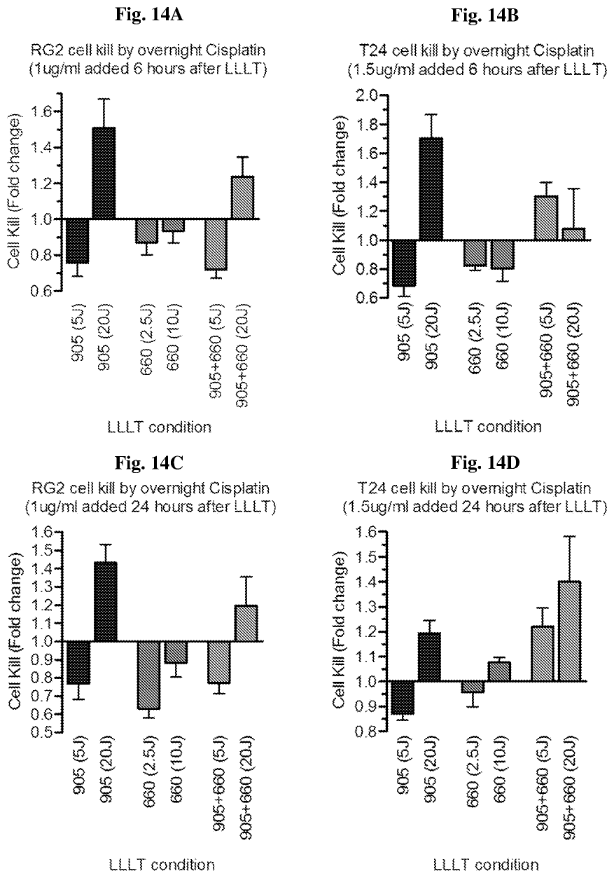

RG2 cells (15,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate. Cells were treated with either 660 nm, 905 nm or a combination of both wavelengths at the indicated fluence doses in J/cm². After 6 or 24 hours, the cells were treated with 5 Gray (“Gy”) of ionizing radiation (“X-rays”), and the plates were returned to the dark in the incubator overnight. Cell viability was measured by Presto Blue cell viability assay quantifying fluorescence at using a plate reader at excitation emissions of 560/600 nm. Values were normalized to control cells that were kept in the dark and not treated with LLLT. The 6-hour results are shown in FIG. 13A and the 24-hour results are shown in FIG. 13B.

### Examples 9A and 9B: LLLT and Cisplatin/Temozolomide

Cisplatin and Temozolomide (“TMZ”) are two chemotherapeutic drugs that are used in treatment of solid tumors; however, they are administered systemically and induce systemic side effects that may dramatically affect the quality of life. This example shows that phototherapeutic modalities increase the efficacy of chemotherapeutic drugs, allowing lower doses to be used, which would significantly decrease the associated side effects.

### Example 9A

RG2 cells (15,000 cells) or T24 cells (10,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate. Cells were treated with either 660 nm, 905 nm or a combination of both wavelengths at the indicated fluence doses in J/cm². After 6 or 24 hours, the cells were treated with Cisplatin (1 ug/ml for RG2 cells or 1.5 ug/ml for T24 cells), and the plates were returned to the incubator overnight. Cell viability was measured by Presto Blue cell viability assay quantifying fluorescence at using a plate reader at excitation emissions of 560/600 nm. Values were normalized to control cells that were kept in the dark and not treated with LLLT. The results are shown in FIGS. 14A-14D.

The data reveal that LLLT has a biphasic effect on enhancing anti-cancer treatment through Cisplatin. Cisplatin-mediated cell kill was increased in cells treated with 905 nm PW or combined 905 nm PW and 660 nm CW at the fluence value of 20 J/cm²; whereas, the lower fluence values of 5 J/cm² for 905 nm PW and for most conditions with 660 nm CW alone promoted cell survival. The data presented in FIGS. 14A-14D suggests that LLLT performed before Cisplatin treatment affects the efficacy of the treatment and knowledge of the treatment dose is essential to avoid any adverse effects; therefore, LLLT can be used to potentiate the efficacy of Cisplatin antineoplastic treatment and allow for decreased drug doses to minimize toxicity of the conducted anti-cancer therapy.

### Example 9B

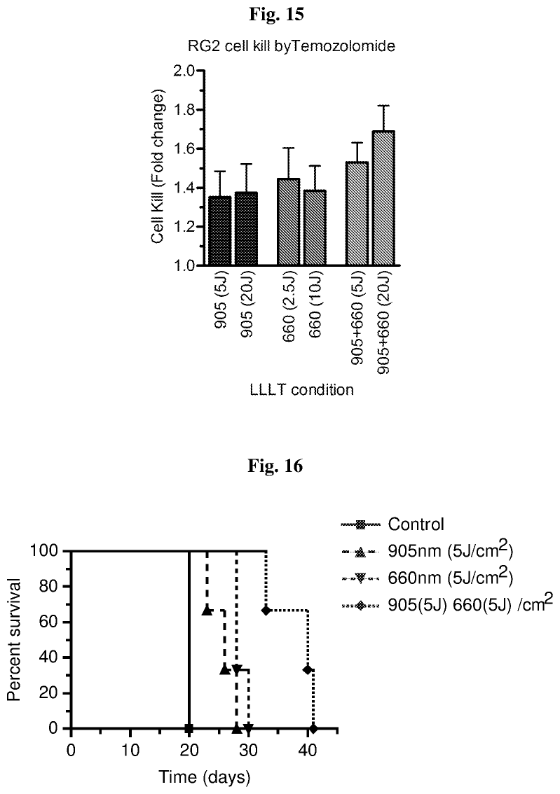

RG2 cells (15,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate containing either DMSO or 1.5 mM TMZ. The next day, the cells were treated with either 660 nm, 905 nm or a combination of both wavelengths at the indicated fluence doses in J/cm² and the plates were returned to the incubator overnight. Cell viability was measured by Presto Blue cell viability assay quantifying fluorescence at using a plate reader at excitation emissions of 560/600 nm. Values were normalized to control cells that were kept in the dark and not treated with LLLT. The results are shown in FIG. 15.

The data revealed that LLLT at all conditions potentiated anti-cancer treatment through the antineoplastic drug TMZ. TMZ-mediated cell kill was increased with cells treated with 905 nm PW, 660 nm CW, or a combination of both wavelengths at the energy doses indicated; therefore, LLLT can potentiate the efficacy of TMZ antineoplastic treatment with a large margin of safety without adverse effects of LLLT observed and would hence allow for decreased drug dose to minimize the toxicity associated with the conducted anti-cancer therapy.

### Example 10: LLLT In Vivo Delays Tumor Growth/Progression

CT26WT (human colon cancer—wild type) tumor cells were implanted in the hind leg of Balb/c mice to develop a hind leg tumor model. When the tumor size reached a size of approximately 5 mm by 5 mm, LLLT treatment was initiated and the mice were treated with either 660 nm, 905 nm or a combination of both wavelengths at the fluence doses of 2.5-20 J/cm² daily for 5 consecutive days. Tumor size and survival were determined. The results show that LLLT increased the survival of mice harboring hind leg tumors by decreasing tumor size (FIG. 16).

### Example 11: LLLT and PDT for In Vivo Hind Leg Tumor Model

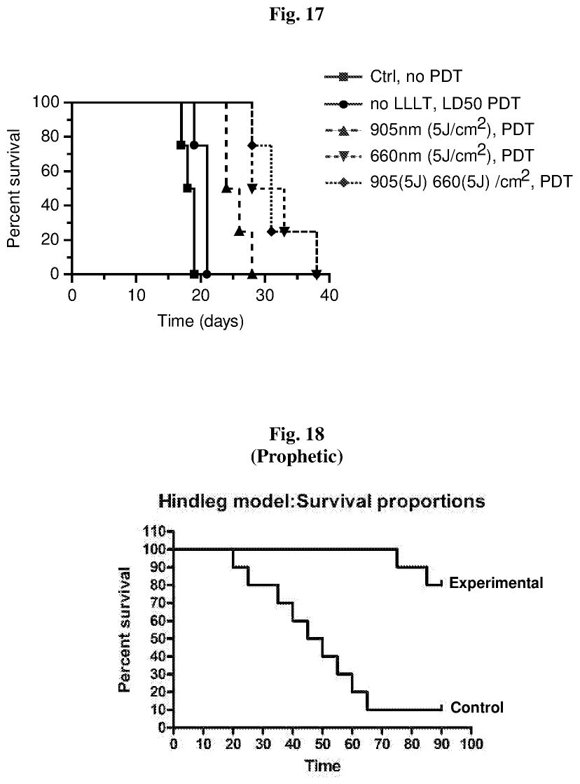

CT26WT tumor cells were implanted in the hind leg of Balb/c mice to develop a hind leg tumor model. When the tumor size reached a size of approximately 5 mm by 5 mm, the LLLT treatment was initiated and mice were treated with either 660 nm, 905 nm or a combination of both wavelengths at the energy dose of 2.5-20 J/cm² daily for 5 consecutive days. TLD-1433 (14C) was injected on day 5 of LLLT treatment and suboptimal red light PDT (90 J/cm²) was performed 4 hours after injection. Tumor size and survival were determined to examine the effect of LLLT on potentiating the effect of 14C-mediated PDT and improving the survival curve. The results show that LLLT and PDT synergistically increased the survival of mice treated with PDT harboring hind leg tumors compared to control mice treated with PDT only (FIG. 17).

### Example 12: LLLT and X-ray for In Vivo Hind Leg Tumor Model (Prophetic)

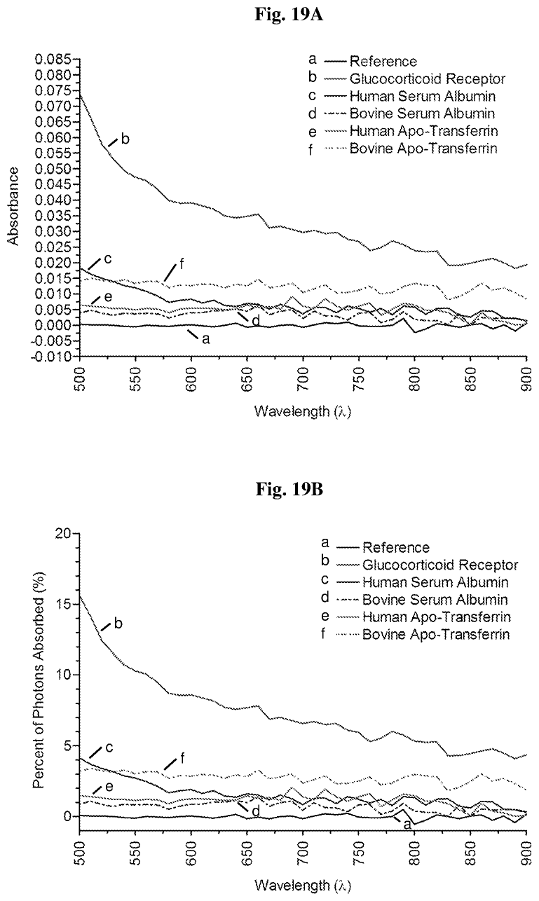

CT26WT tumor cells are implanted in the hind leg of Balb/c mice to develop a hind leg tumor model. When the tumor size reaches a size of approximately 5 mm by 5 mm, LLLT procedure is initiated and mice are treated with either 660 nm, 905 nm or a combination of both wavelengths at an energy dose between 2.5-20 J/cm² daily for 5 consecutive days. Suboptimal X-ray ionizing radiation (1 Gray) over the area of the tumor location is performed. Tumor size and survival are determined to examine the effect of LLLT on potentiating the effect of tumor cell kill by X-ray and improving the survival curve. Data will show that LLLT and X-ray synergistically increase the survival of mice treated with hind leg tumors versus mice with hind leg tumors treated by X-ray only (FIG. 18).

### Example 13: Absorbance of GCR as a Potential Photoreceptor for LLLT

Enhanced susceptibility to inflammatory, degenerative, metabolic, behavioral, age-related and/or autoimmune diseases may be related to impairments in HPA axis activity and associated hypocortisolism, or to Gluco Corticoid (“GC”) resistance resulting from impairments in local factors affecting GC availability and function, including the GCR. The enhanced inflammation and hypercortisolism that typically characterize stress-related illnesses, such as: depression, metabolic syndrome, cardiovascular disease or osteoporosis, may also be related to increased GC resistance; hence, LLLT may be used in the Warburg effect-mediated and the GCR impairment (function)-mediated conditions. The evidence that GCR function and GC resistance can be modulated by LLLT may have important implications for management of stress-related inflammatory illnesses and underscores the importance of prevention and management of chronic stress.

The GCR is a member of the nuclear hormone receptor superfamily and plays a critical role in metabolism, development, reproduction and/or homeostasis. The GCRs are found in the cytoplasmic fraction from which they can be isolated. Cytosolic changes due to LLLT have been observed that are not explainable via the LLLT-mitochondrial pathway. LLLT targeting of bioenergetic (Warburg) pathways via or in combination with GCR pathways may offer a promising therapeutic strategy to overcome anti-cancer resistance and optimize the therapeutic mechanisms of LLLT.

Recombinant GC-protein was purchased from Thermofisher Scientific pre-dissolved in a 10 mM potassium phosphate buffer (pH=7.4) storage buffer containing 5 mM Dithiothreitol (“DTT”), 10 mM sodium molybdate, 10% glycerol and 0.1 mM Ethylenediaminetetraacetic Acid (“EDTA”). Accordingly, a similar storage buffer was prepared for background reference absorption readings. In addition, other proteins (i.e., human and bovine serum albumin and human and bovine Apo-transferrin) were prepared at the same molar concentration in the same storage buffer as GC. Three independent absorbance measurement experiments were performed on two different spectrophotometers (Spectramax Pro5 and Ultraspec 2100 Pro) at 5 nm intervals between 500 and 900 nm wavelengths and reference background values were subtracted to account for light absorbance by the dissolving buffer. Percentage of photons absorbed was calculated using the following formula: Photons Absorbed=(1-10-Abs)*100

As shown in FIGS. 19A and 19B, the data revealed that the GC has significantly higher absorbance than background reference and other proteins at most wavelengths, most notably: in the green, red, and near infrared spectrums.

### Example 14: Effect of LLLT on Hexose-6-Phosphate Dehydrogenase (“H6PD”) Enzyme Activity

Circulating GC levels are controlled by the Hypothalamo-Pituitary-Adrenal (“HPA”) axis, but within tissues, GC availability is controlled by 11β (Beta)-Hydroxysteroid Dehydrogenase (“11β (Beta)-HSD”) 11β (Beta)-HSD converts inactive cortisone to active cortisol. Dysregulated 110 (Beta)-HSD activity has been implicated in many metabolic diseases such as obesity and diabetes and inhibition of 11β (Beta)-HSD represents a promising therapeutic target. Hexose-6-phosphate dehydrogenase (“H6PD”) has emerged as an important factor in setting the redox status of the Endoplasmic Reticulum (“ER”) lumen. An important role of H6PD is to generate a high NADPH/NADP+ ratio which permits 11β (Beta)-HSD to act as an oxo-reductase, catalyzing the activation of GCs. In order to prove this hypothesis, we measured the effect of LLLT on H6PD activity as a measure of the effect of LLLT on GR signaling.

RG2 (0.6×10⁶ cells) and T24 ((0.4×10⁶ cells) were seeded in 6 cm culture plates in complete DMEM and incubated at 37° C., 5% CO₂ overnight. When the cells reached 90% confluency, the plates were washed once with PBS and the medium was replaced with complete DMEM without phenol red or sodium pyruvate. Cells were treated with either 660 nm, 905 nm or a combination of both wavelengths at the indicated energy doses in J/cm². After 2 hours, the cells were collected by scraping with a rubber cell scraper in cold 1×PBS. Cells were sonicated on ice twice for 20 seconds each separated by a 30 second interval on ice, and the lysate was centrifuged at 10,000 g for 10 min at 4° C. 10 ul from the supernatant was used to determine H6PD activity using the Glucose-6-phosphate dehydrogenase (“G6PDH”) assay kit (Cayman Chemicals) with using 2-deoxyglucose-6-phosphate (“2DG-6P”) as the substrate instead of glucose-6-phosphate (“G6P”) substrate supplied with the kit. The plate was incubated for 20 min at 37° C. and fluorescence was quantified using a plate reader at excitation emissions of 535/590 nm. Values represent fold differences relative to values from dark control cells (not treated with LLLT), set as 1. *p<0.05; **p<0.01; ***p<0.001. n=6 from 3 independent experiments.

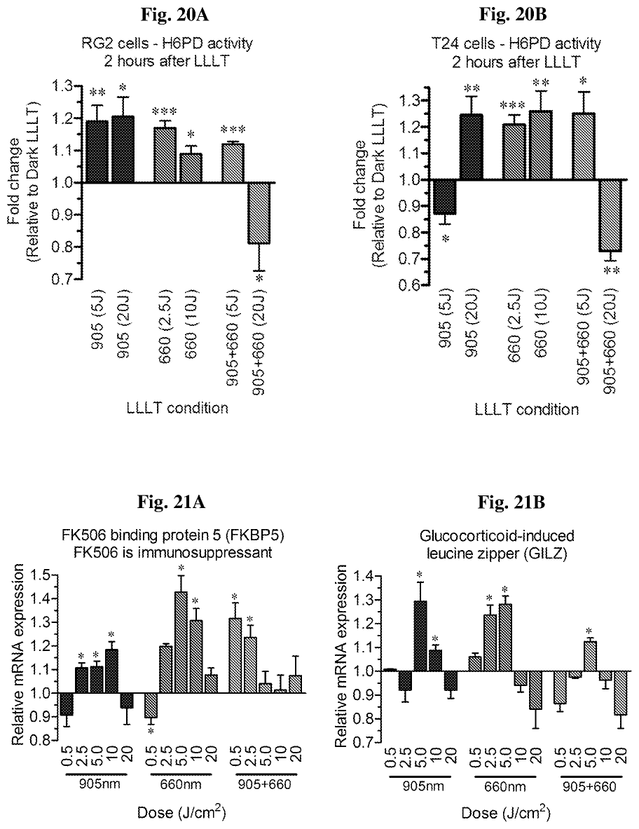

As shown in FIGS. 20A and 20B, the data revealed that LLLT increases H6PD enzyme activity by approximately 20%, which would result in increased NADPH production as a byproduct, which in turn would result in increased activity of 11β-hydroxy-steroid dehydrogenase (“11β-HSD”) enzyme and increased conversion and activation of GC into its active form (i.e., cortisone to cortisol). Cortisol binds to the GCR resulting in: 1) direct effects on gene expression in the nucleus through GCR-response elements; 2) effects on mitochondrial genes and metabolism through GCR in mitochondrial DNA; 3) binding to and repressing other transcription factors (e.g., NF-kB, AP-1, etc.). These results indicate that LLLT may affect GCR signaling through activation of H6PD and 11β-HSD enzymes activity.

### Example 15: Effect of LLLT on GCR Downstream Canonical Signaling Targets

RG2 cells (0.5×10⁶ cells) were seeded in complete DMEM in 35 mm culture dishes and incubated at 37° C., 5% CO₂ overnight. The next day, the cells were washed once with PBS and the medium was replaced with complete DMEM without phenol red or sodium pyruvate. Cells were treated with either 660 nm, 905 nm or a combination of both wavelengths at energy doses from 0.5 to 20 J/cm². Cells were incubated for 6 hours, then the cells were washed once with PBS and harvested by TRIzol reagent for RNA extraction (Invitrogen) per the manufacturer's protocol. 2 ug of RNA were used to prepare cDNA using the high-capacity cDNA reverse transcription kit (Thermofisher) and the cDNA was diluted 1:10. 2 ul of cDNA was used per PCR reaction using the SYBR green master mix and qPCR was run on the LightCycler 480 (Roche). Gene expression was normalized to the housekeeping gene HPRT and values were normalized to control cells that were kept in the dark and not treated with LLLT.

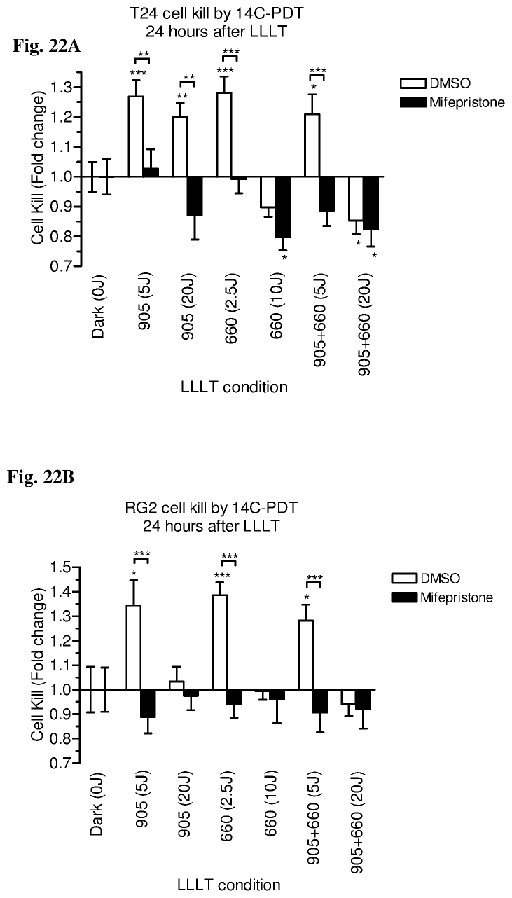

As shown in FIGS. 21A and 21B, LLLT mediated the effects via regulation of indigenous steroids (hypothalamic-pituitary-adrenal pathway), but also significantly upregulated the level of indigenous tacrolimus—FK506. Tacrolimus is known to reduce the activation of macrophages/microglia (“APC”) in-vitro and/or in-vivo and affects the expression of various cytokines like interleukin-1, interleukin-6 and/or tumor necrosis factors, as well as cell mediated inflammation.

### Example 16: Influence of GCR Agonists/Antagonists on LLLT-Effect on Warburg Glycolysis (Prophetic)

RG2 cells (30,000 cells) or human bladder cancer (T24) cells (20,000 cells) are seeded in complete DMEM in 96-well Seahorse culture plates (Agilent technologies) and incubated at 37° C., 5% CO₂. The cells are treated for 24 hours with the GCR agonist, dexamethasone, or the GCR antagonist, Mifepristone (10 uM). The cells are then washed once with PBS and the medium replaced with complete DMEM without phenol red or sodium pyruvate. Cells are treated with either 660 nm, 905 nm or a combination of both wavelengths at energy doses from 0.5 to 10 J/cm². Cells are incubated for 24 hours. The media is then replaced with XF media without sodium bicarbonate and the plate placed at 37° C. in XF prep station (0% CO₂) for 1 hour. Basal ECAR and OCR levels are measured using XFe96 analyzer (Agilent technologies) per the manufacturer's protocol. The GCR agonist, dexamethasone, inhibits the Warburg effect by decreasing the glycolysis rate and increasing mitochondrial respiration; whereas, the GCR antagonist Mifepristone potentiates the Warburg effect by increasing glycolysis and decreasing mitochondrial respiration. Moreover, the GCR antagonist, Mifepristone, reverses the positive effect that LLLT has on inhibiting the Warburg effect.

### Example 17: Effect of GCR Inhibitor Mifepristone on LLLT-Mediated Synergy with PDT In-Vitro

RG2 cells (7,500 cells) or human bladder cancer (T24) cells (5,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate, with or without 10 uM GCR antagonist mifepristone for an additional 24 hours. Cells were treated with either 660 nm, 905 nm or a combination of both wavelengths at the indicated energy doses in J/cm². After 6 or 24 hours, the media was replaced with media containing the ruthenium-based photodynamic compound 14C (100 nM for RG2 cells and 200 nM for T24 cells) for 3 hours, then the drug was washed away and the cells were treated with PDT using green laser (wavelength: 530 nm, fluence 20 J/cm²) using a 96-laser diode array light source (TLD-3000, Theralase Inc. Toronto, ON, Canada), and the plates were returned to the dark in the incubator overnight. Cell viability was measured by Presto Blue cell viability assay quantifying fluorescence at using a plate reader at excitation emissions of 560/600 nm.

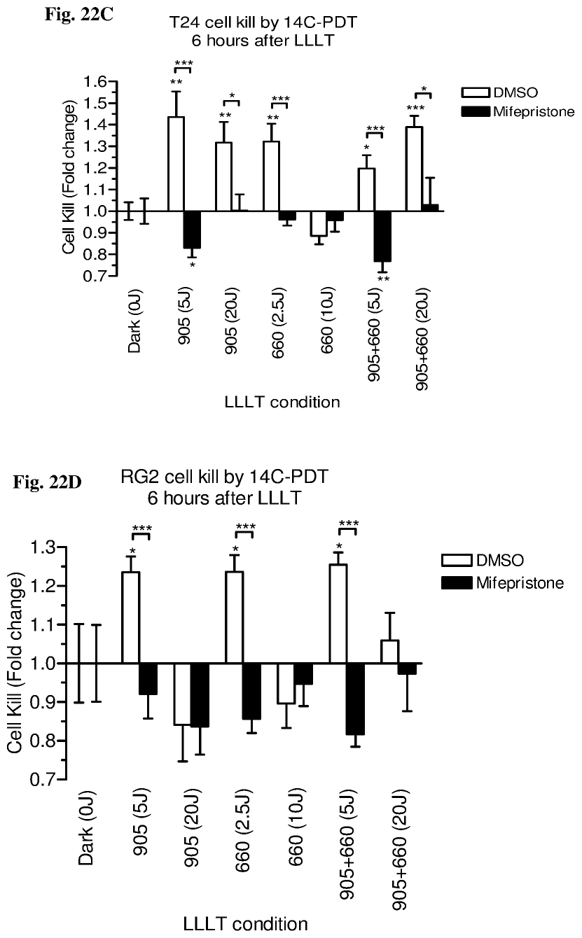

As shown previously, when LLLT is combined with PDT (without Mifepristone pre-treatment, white bars), LLLT increased the PDT effect when cells were treated with 905 nm (5 J/cm²), 660 nm (2.5 J/cm²), or a combination of 905 nm and 660 nm (5 J/cm²); whereas, the higher doses (10-20 J/cm²) induced a biphasic effect and decreased the PDT effective cell kill. p values above the white bars indicate significant differences compared to the dark LLLT control (set as 1). See FIGS. 22A-22D.

Mifepristone treatment alone did not induce cell kill in T24 and RG2 cells and Mifepristone treatment did not have any significant effects on PDT cell kill; therefore, LLLT combined with PDT with Mifepristone pre-treatment (black bars) was normalized to the corresponding dark LLLT control group (set as 1), and p values above the black bars indicate significant differences compared to this group.

Significant differences in cell kill (for each LLLT condition) between DMSO- or Mifepristone-treated cells (white versus black bars) is indicated by brackets. Overall, Mifepristone pre-treatment significantly inhibited the synergistic effect of LLLT on 14C PDT. For example, Mifepristone blocked the increase in cell kill in cells treated with LLLT (905 (5 J/cm²), 660 nm (2.5 J/cm²), or a combination of 905 nm and 660 nm (5 J/cm²).

### Example 18: Effect of GCR Inhibitor Mifepristone on LLLT Synergy with Radiation Therapy by X-ray (Prophetic)

RG2 cells (7,500 cells) or human bladder cancer (T24) cells (5,000 cells) are seeded in complete DMEM in 96-well culture plates and incubated at 37° C., 5% CO₂ overnight. The next day, the cells are washed once with PBS and the medium is replaced with complete DMEM without phenol red or sodium pyruvate, with or without 10 uM GCR antagonist, mifepristone, for another 24 hours. Cells are treated with either 660 nm, 905 nm or a combination of both wavelengths at the indicated energy doses in J/cm². After 1 or 24 hours, the cells are treated with 5 Gy of X-rays and the plates are returned to the dark in the incubator overnight. Cell viability is measured by Presto Blue cell viability assay quantifying fluorescence at using a plate reader at excitation emissions of 560/600 nm.

### Example 19: Effect of GCR Inhibitor Mifepristone on LLLT Synergy with Antineoplastic Drugs Cisplatin

RG2 cells (7,500 cells) or human bladder cancer (T24) cells (5,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate, with or without 10 uM GCR antagonist, Mifepristone, for another 24 hours. Cells were treated with either 660 nm, 905 nm or a combination of both wavelengths at the indicated energy doses in J/cm². After 6 or 24 hours, the cells were treated with Cisplatin (1 ug/ml for RG2 cells or 1.5 ug/ml for T24 cells), and the plates were returned to the incubator overnight. Cell viability was measured by Presto Blue cell viability assay quantifying fluorescence at using a plate reader at excitation emissions of 560/600 nm. Values will be normalized to control cells that are kept in the dark and not treated with LLLT.

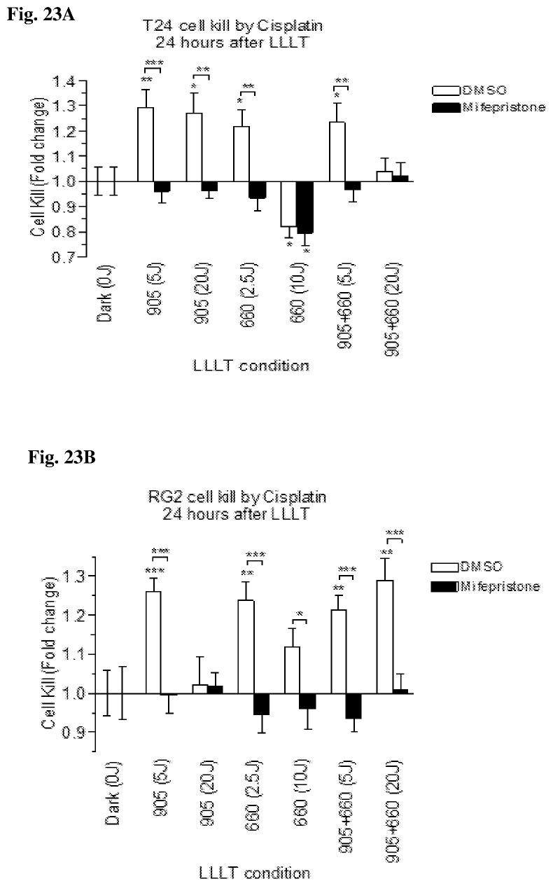

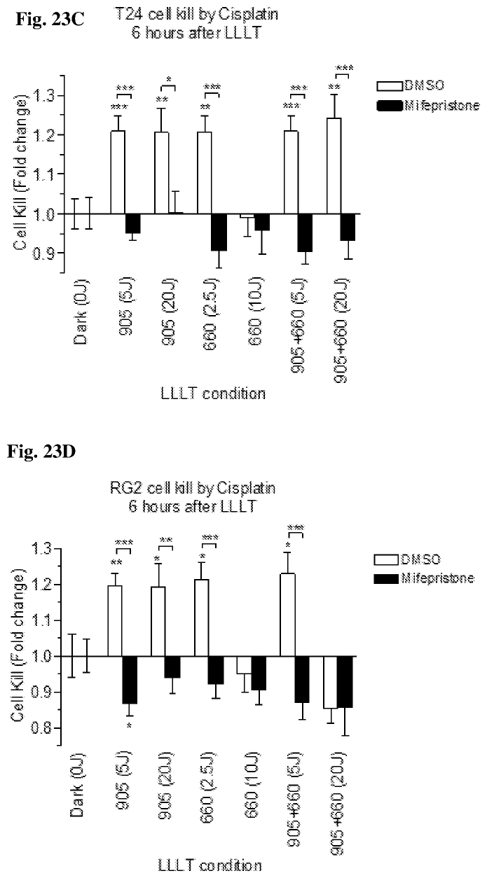

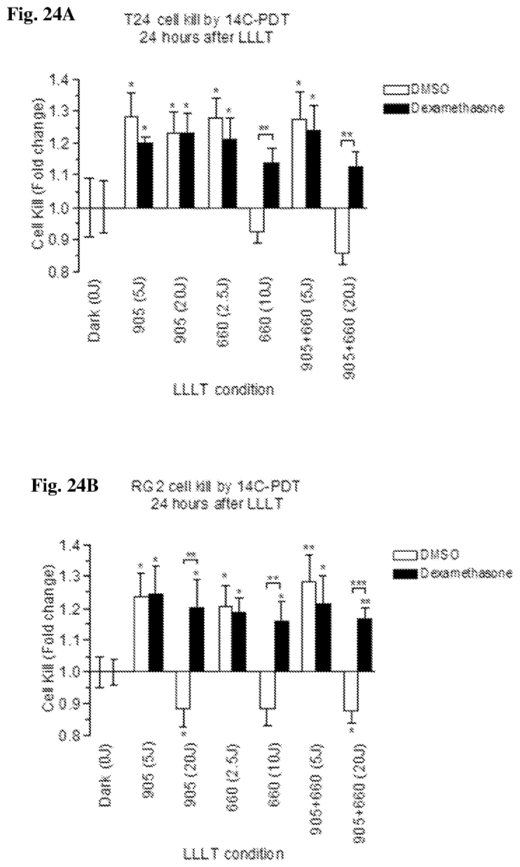

The results are similar to the previous observations with PDT. See FIGS. 23A-23D. As shown previously, when LLLT is combined with Cisplatin (without Mifepristone pre-treatment, white bars), LLLT increased the Cisplatin effect when cells were treated with 905 nm (5 J/cm²), 660 nm (2.5 J/cm²), or a combination of 905 nm and 660 nm (5 J/cm²); whereas the higher doses (10-20 J/cm²) induced a biphasic effect and decreased the Cisplatin effective cell kill. p values above the white bars indicate significant differences compared to the dark LLLT control (set as 1).

Mifepristone treatment alone did not induce cell kill in T24 and RG2 cells and Mifepristone treatment did not have any significant effects on Cisplatin cell kill; therefore, LLLT combined with Cisplatin and with Mifepristone pre-treatment (black bars) was normalized to the corresponding dark LLLT control group (set as 1), and p values above the black bars indicate significant differences compared to this group.

Significant differences in cell kill, for each LLLT condition, between DMSO or Mifepristone treated cells (white versus black bars) is indicated by brackets. Overall, Mifepristone pre-treatment significantly inhibited the synergistic effect of LLLT on Cisplatin. For example, Mifepristone blocked the increase in cell kill in cells treated with LLLT (905 (5 J/cm²), 660 nm (2.5 J/cm²) or a combination of 905 nm and 660 nm (5 J/cm²).

### Example 20: Effect of GCR Agonist Dexamethasone on PDT, X-ray Radiation, and Antineoplastic Drugs

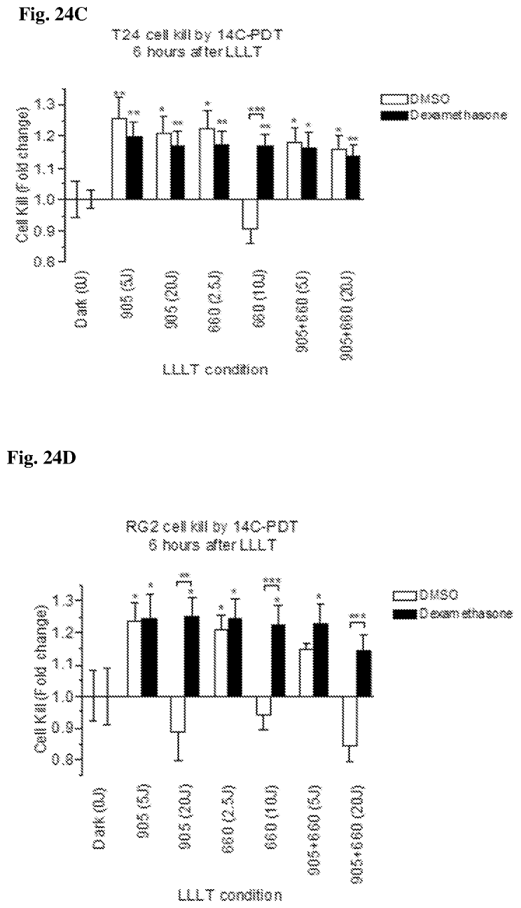

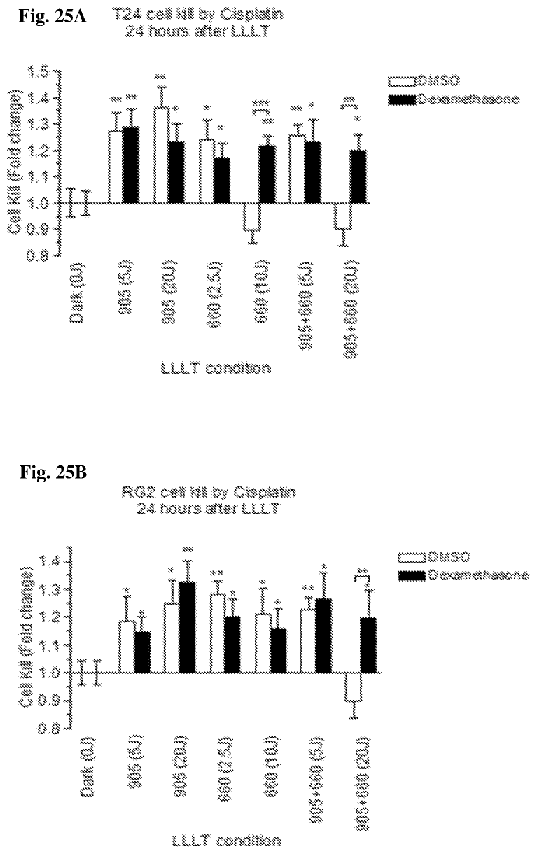

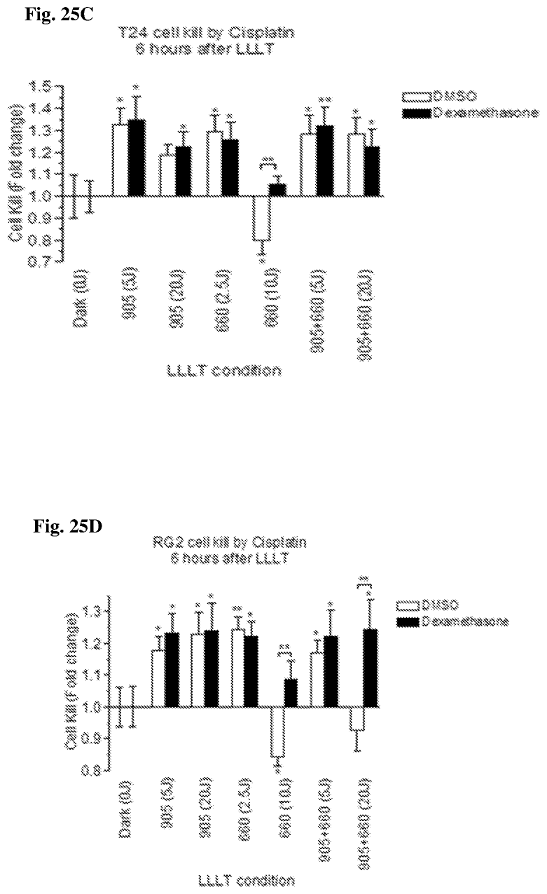

RG2 cells (15,000 cells) or human bladder cancer (T24) cells (10,000 cells) were seeded in complete DMEM in 96-well culture plates and incubated at 37° C., 5% CO₂ overnight. The next day, the cells were washed once with PBS and the medium was replaced with complete DMEM without phenol red or sodium pyruvate, with or without 1 uM GCR agonist, dexamethasone, for another 24 hours. Cells were then treated with either: 1) the ruthenium-based PDC 14C followed by PDT using a green laser light source (FIGS. 24A-24D); 2) the antineoplastic drug Cisplatin (FIGS. 25A-25D). The plates were then returned to the incubator overnight. Cell viability was measured by Presto Blue cell viability assay quantifying fluorescence at using a plate reader at excitation emissions of 560/600 nm. Values were normalized to control cells that were not treated with dexamethasone.

When LLLT had a positive effect on 14C-PDT or Cisplatin cell kill, the agonist did not have a synergistic or an additive effect and the positive effect remained the same with no significant differences, but when LLLT had a negative effect with higher LLLT fluence (10-20 J/cm²), dexamethasone reversed that effect to a positive significantly different effect. The “*” above each bar represents significant differences compared to the corresponding dark control group (set as 1) and significant differences between DMSO or DEX are indicated by brackets above white and black bars (*p<0.05, **p<0.01, ***p<0.001).

### Example 21: Effect of LLLT on the Stem-Like Characteristics of Cancer Cells in a GCR-Dependent Mechanism

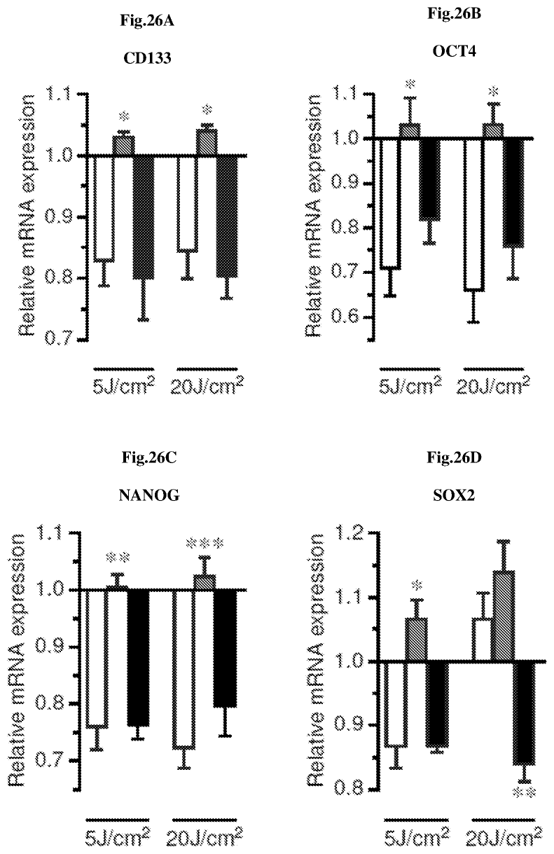

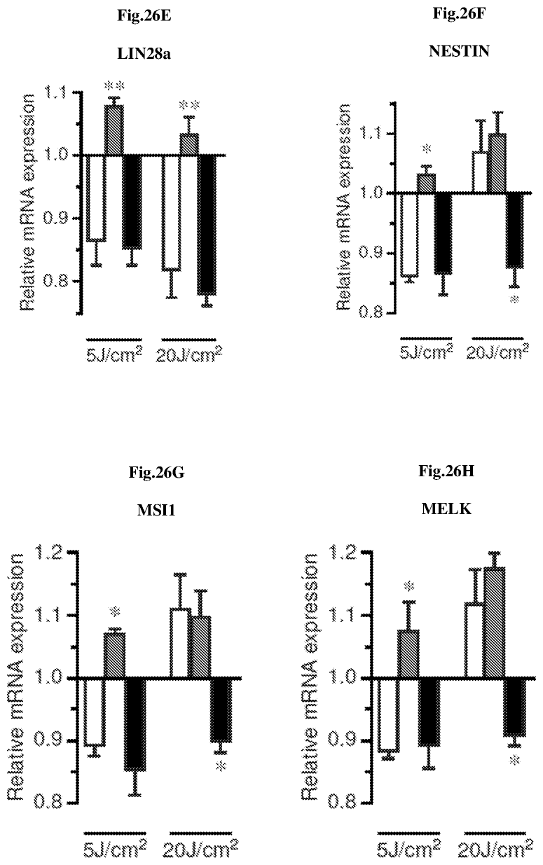

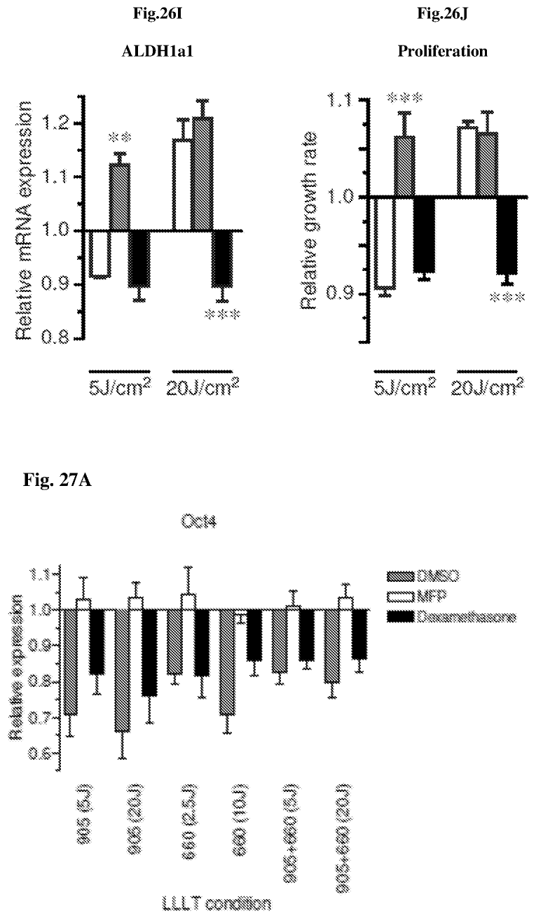

RG2 cells (0.5×10⁶ cells) were seeded in complete DMEM in 35 mm culture dishes and incubated at 37° C., 5% CO₂ overnight. The next day, the cells were washed once with PBS and the medium was replaced with complete DMEM without phenol red or sodium pyruvate containing vehicle (DMSO, white bars of FIGS. 26A-26J), GCR antagonist (Mifepristone, grey bars of FIGS. 26A-26J) or GCR agonist (Dexamethasone, black bars of FIGS. 26A-26J) and plates were incubated for 24 hours. Cells were then treated with 905 nm at fluence doses of 5 or 20 J/cm². Cells were incubated for 6 hours, then the cells were washed once with PBS and harvested by TRIzol reagent for RNA extraction (Invitrogen) per the manufacturer's protocol. 2 ug of RNA were used to prepare cDNA using the high-capacity cDNA reverse transcription kit (Thermofisher) and the cDNA was diluted 1:10. 2 μl of cDNA was used per PCR reaction using the SYBR green master mix and qPCR was run on the LightCycler 480 (Roche). Gene expression was normalized to the housekeeping gene HPRT, and values were normalized to control cells that were kept in the dark and not treated with LLLT. Relative analysis of stem cell associated transcription factor genes CD133, OCT4, NANOG, SOX2 and LIN28a, or proliferation promoting genes NESTIN, MSI1, MELK and ALDH1a1.

FIG. 26J shows an analysis of the proliferation rate defined as the inverse of T (time when cells reach confluency halfway between original seeding density (15%) and final 100% confluency). RG2 cells (5,000 cells) were seeded in complete DMEM without phenol red or sodium pyruvate in 96-well culture plate and incubated at 37° C., 5% CO₂. Phase contrast images (4×) were taken every 4 hours using the Incucyte ZOOM system to calculate percent confluency overtime. Cells were pre-treated with DMSO, 10 uM GCR antagonist Mifepristone, or 20 uM GCR agonist Dexamethasone and media was changed daily. Cells were treated with daily either 660 nm, 905 nm, or a combination of both wavelengths at the indicated energy dose (J/cm²). Proliferation rate was calculated as the inverse of T (time when cells reach confluency halfway between original seeding density (15%) and final 100% confluency). Data were normalized to the corresponding dark control group (0 J/cm²) for each drug expressed as 1 (not shown). Data were normalized to the corresponding dark control group (0 J/cm²) for each drug expressed as 1 (data not shown). Data (mean and SEM) are from three independent experiments. *P<0.05, **P<0.01 and ***P<0.001 (one-way ANOVA (P<0.0001) with Tukey's post-hoc test, relative to DMSO control).

These results show that LLLT has biphasic effect on stem-like characteristics of cancer cells and that these changes are dependent on GCR function. Positive effects of LLLT were blocked by Mifepristone pre-treatment, and negative biphasic effects of LLLT were reversed by Dexamethasone pre-treatment. Thus, a nuclear receptor agonist, particularly a glucocorticoid receptor agonist, can modulate the sternness of cancer stem cells enabling differentiation of stem cells and hence more effective anticancer therapies.

### Example 22: Effect of LLLT on the Stem-Like Characteristics of Cancer Cells in a GCR-Dependent Mechanism

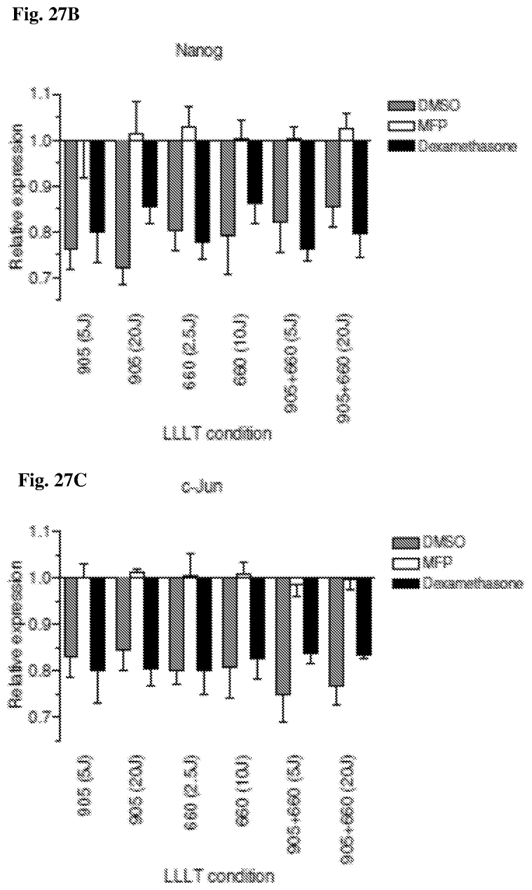

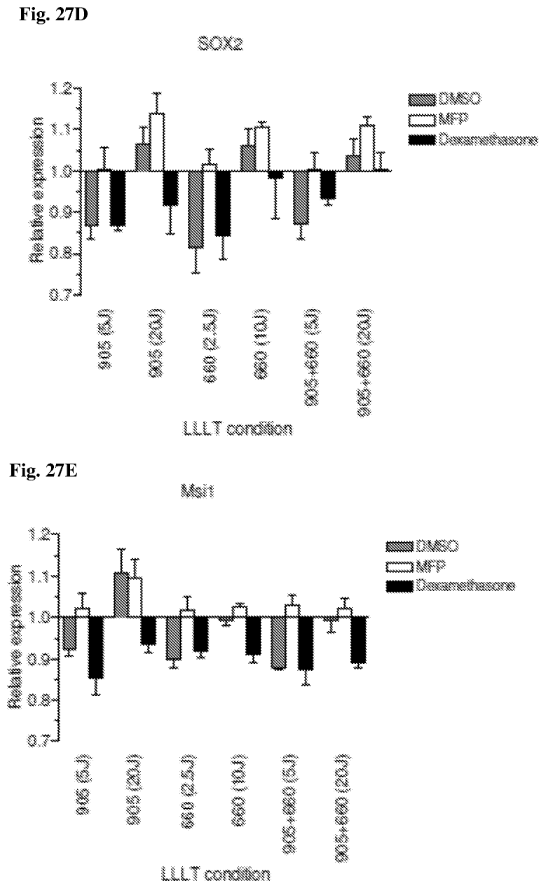

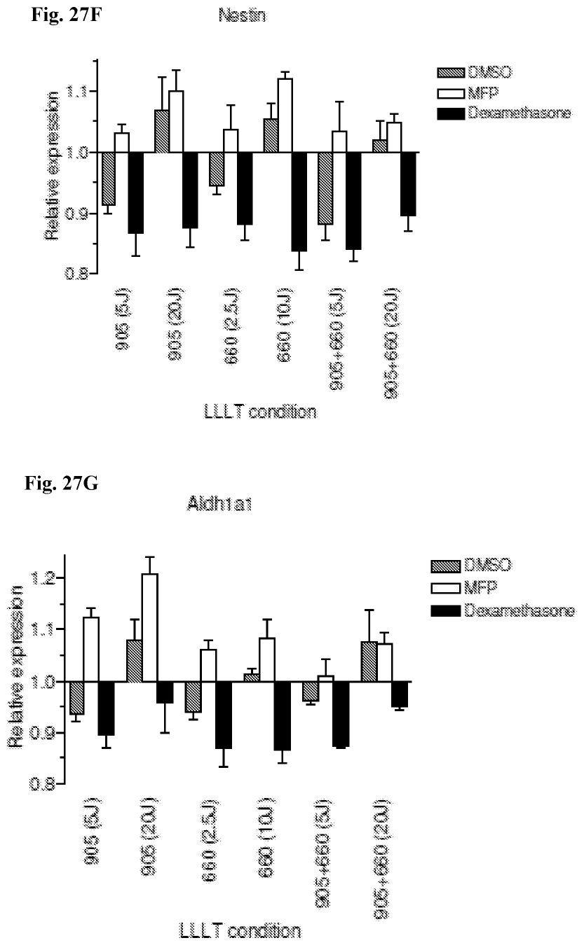

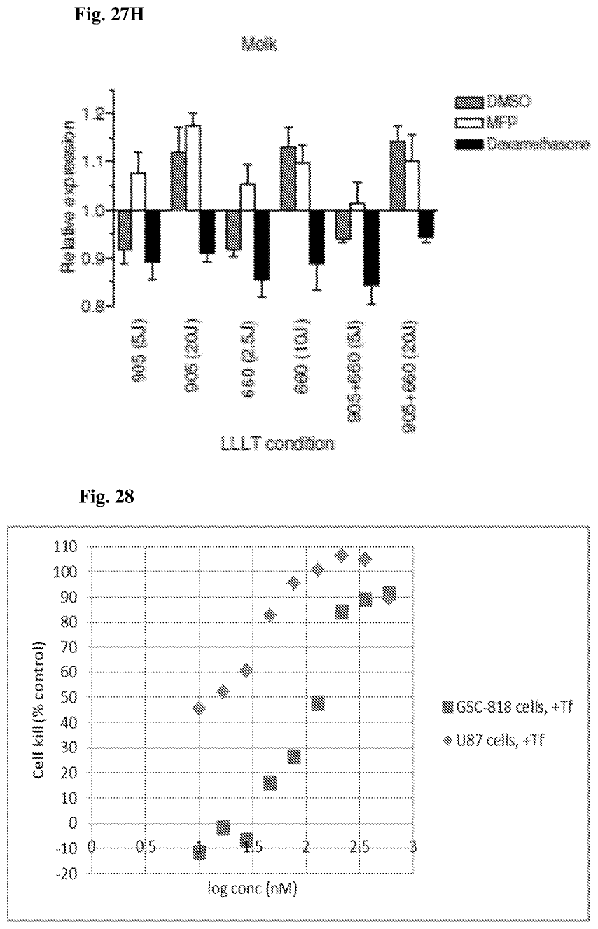

RG2 cells (0.5×10⁶ cells) were seeded in complete DMEM in 35 mm culture dishes and incubated at 37° C., 5% CO₂ overnight. The next day, the cells were washed once with PBS and the medium was replaced with complete DMEM without phenol red or sodium pyruvate containing vehicle (DMSO, grey bars of FIGS. 27A-27H), GCR antagonist (Mifepristone, white bars of FIGS. 27A-27H) or GCR agonist (Dexamethasone, black bars of FIGS. 27A-27H) and plates were incubated for 24 hours. Cells were then treated with either 660 nm light, 905 nm light or a combination of both wavelengths at fluence doses from 2.5 or 20 J/cm². Cells were incubated for 6 hours, then the cells were washed once with PBS and harvested by TRIzol reagent for RNA extraction (Invitrogen) per the manufacturer's protocol. 2 ug of RNA were used to prepare cDNA using the high-capacity cDNA reverse transcription kit (Thermofisher) and the cDNA was diluted 1:10. 2 μl of cDNA was used per PCR reaction using the SYBR green master mix and qPCR was run on the LightCycler 480 (Roche). Gene expression was normalized to the housekeeping gene HPRT, and values were normalized to control cells that were kept in the dark and not treated with LLLT.

FIGS. 27A-27F show relative analysis of stem cell associated transcription factor genes OCT4, NANOG, SOX2 and c-JUN, or proliferation promoting genes NESTIN, MSI1, MELK and ALDH1a1.

FIG. 28 shows stem cells (GSC-818), a human glioma cell line, are more resistant to PDT than non-stem cells (U87), a differentiated human glioma (brain cancer) cell line, with greater LD50 for pure PDT effect (LD50=115.9 nM, CI95=86.12-170.4 nM) than U87 cells (LD50=20.16 nM, CI95=11.37-34.07 nM). CONDITIONS: PDT @ 530 nm, 20 Jcm-2, 3:1 Rutherrin, 4 hours loading time, not washed during PDT and until Presto Blue staining, 1 day endpoint).

While the invention has been described in detail and with reference to specific examples thereof, it will be apparent to one skilled in the art that various changes and modifications can be made therein without departing from the spirit and scope thereof.

1. A method of activating a nuclear receptor in an organism, said method comprising irradiating the nuclear receptor with light effective to activate the nuclear receptor to cause a biological effect in the organism, wherein the nuclear receptor is not specific to a retina of the organism.

2. The method of claim 1, wherein the biological effect is an influence exerted on at least one pathway selected from the group consisting of a hypothalamic-pituitary-adrenal pathway, a renin-angiotensin pathway, a vagal-neuronal pathway and a neuro-hormonal-immunal pathway.

3. The method of claim 1, wherein the biological effect is an influence on cellular metabolism.

4. The method of claim 1, wherein the biological effect is an alteration in a response of the organism to a different treatment modality.

5. The method of claim 4, wherein the different treatment modality is selected from the group consisting of ionizing radiation therapy, photo dynamic therapy, surgical intervention, chemotherapy and sonodynamic therapy.

6. The method of claim 1, wherein the biological effect is an alteration of a Warburg effect so as to make cells more susceptible to treatment and/or prophylaxis of a disorder.

7. The method of claim 6, wherein the disorder is selected from the group consisting of a mitochondrial disorder, a cardiovascular disorder, a metabolic disorder, an inflammatory disorder, an immune disorder, a degenerative disorder, muscle fatigue, an aging disorder and cancer.

8. The method of claim 1, wherein the nuclear receptor is not RNR or NR2E3.

9. The method of claim 1, wherein the biological effect is treating or preventing a disorder in the organism, which is a mammal.

10. The method of claim 9, wherein the light is laser light applied in at least one mode selected from the group consisting of a continuous wave mode, a pulsed mode or a superpulsed mode.

11. The method of claim 10, wherein the light has a photon energy less than 10 eV.

12. The method of claim 10, wherein the light has a photon energy of 9.99-1.23 eV.

13. The method of claim 10, wherein the light has a photon energy of 1.23-0.411 eV.

14. The method of claim 10, wherein 10-90% of photons of the light have a photon energy of 9.99-1.23 eV.

15. The method of claim 10, wherein 10-90% of photons of the light have a photon energy of 1.23-0.411 eV.

16. The method of claim 10, wherein the light is administered at frequencies at or above 10,000 Hz or below 10,000 Hz.

17. The method of claim 10, wherein the light is applied in a patient-specific protocol.

18. The method of claim 10, further comprising administering to the organism at least one member selected from the group consisting of X-rays, magnetic stimuli, electrical stimuli, ultrasound and medicaments.

19. The method of claim 18, wherein the medicaments are photo dynamic compounds, cytotoxic drugs, steroids, hormones, sirolimus, tacrolimus, anti-inflammatories, immunomodulators, vitamins or vitamin analogues.

20. The method of claim 10, wherein light of specific wavelengths, modulations and/or energy doses influences functioning of nuclear receptors in a biphasic response, which in turn positively or negatively affects reactions of the organism to stimuli.

21. The method of claim 1, wherein the biological effect mimics an effect of a naturally occurring hormone or a synthetic hormone.

22. The method of claim 1, wherein the biological effect mimics an effect of at least one of sirolimus and tacrolimus.

23. The method of claim 1, wherein the light induces or represses gene expression.

24. The method of claim 1, wherein the biological effect is a non-retinal regulation of circadian rhythm.

25. The method of claim 1, wherein the biological effect is regulation of at least one of calcium and phosphate levels.

26. The method of claim 1, wherein the biological effect mimics an effect of Peroxisome proliferator-activated receptor (“PPAR”) γ modulators.

27. The method of claim 1, wherein the biological effect is regulation of at least one of bone homeostasis, bone formation, tissue remodeling and tissue repair.

28. The method of claim 1, wherein the light is a combination of visible and NIR light and is applied to induce or avoid a biphasic effect.

29. The method of claim 1, wherein the biological effect is a modulation of the stemness of cancer stem cells enabling differentiation of the cancer stem cells in anticancer therapy.

30. (canceled)

31. A laser device configured to perform the method of claim 1.
